(19) United States
(12) Patent Application Publication
(10) Pub. No.: US 2024/0239805 A1
(43) Pub. Date: Jul. 18, 2024

(54) AZA-YANG CYCLIZATION-BUCHNER AROMATIC RING EXPANSION: COLLECTIVE SYNTHESIS OF CYCLOHEPTATRIENE-CONTAINING AZETIDINE LACTONES

(71) Applicant: University of Kansas, Lawrence, KS (US)

(72) Inventors: Manvendra Pal Singh, Lawrence, KS (US); Zarko Boskovic, Lawrence, KS (US); Bryce Gaskins, Lawrence, KS (US)

(21) Appl. No.: 18/541,565

(22) Filed: Dec. 15, 2023

Related U.S. Application Data
(60) Provisional application No. 63/433,397, filed on Dec. 16, 2022.

(51) Int. Cl. C07D 491/20; C07D 471/04

(52) U.S. Cl. CPC C07D 491/20; C07D 471/04

(57) ABSTRACT

The present disclosure is directed to a compound of Formula I, Formula II, Formula III, Formula IV, or Formula V

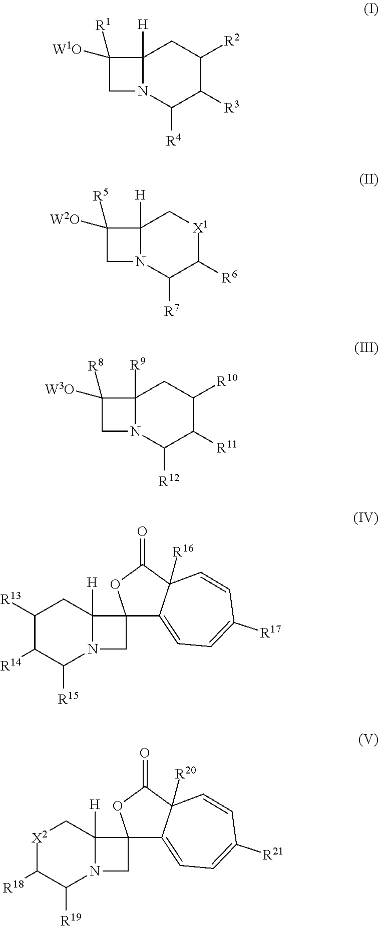

or a pharmaceutically acceptable salt and/or solvate thereof.

### CROSS REFERENCE TO RELATED APPLICATIONS

This application claims the benefit of priority to U.S. Provisional Patent Application No. 63/433,397, filed on Dec. 16, 2022, the entirety of which is incorporated herein by reference.

### U.S. GOVERNMENT RIGHTS

This invention was made with government support under GM113117 and GM136453 awarded by the National Institutes of Health. The government has certain rights in the invention.

### FIELD

The present technology generally relates to complex cycloheptatriene-containing azetidine lactones and methods of preparing them.

### BACKGROUND

Target-oriented organic synthesis is a well-codified endeavor. The stationary nature of the target (often a natural product of considerable structural complexity) and the finality of the effort when the target is reached have contributed to the utility of target-oriented synthesis for probing the capabilities of existing synthetic methods as well as the discovery of new ones, as various researchers execute different pathways en route to a target. Multiple measures of success exist in this realm.¹ Surrounding the field of target-oriented synthesis lies the broader field of diversity-oriented synthesis in which the goal is not a singular target molecule, but rather entirely new molecules inhabiting unexplored chemical space.² Searching for “newness” rather than a specific target molecule leaves one asking questions like the following: How do you plan such a reaction sequence? How do you know when to end? Which concessions to efficiency and selectivity are allowed and which ones are best avoided? For a given end product, what is the measure by which the sequence leading to it can be evaluated? The use of indices of structural complexity may guide structural exploration of chemical space.^{3,4} Using these indices to monitor reactions conducted, or planned, accomplishes two goals: it favors the synthesis of new classes of structures, and it enables the discovery of new synthetic methods and/or the refining of existing ones in new contexts.

In addition to structural complexity, the “information density” of molecules, a notion recently introduced by Shenvi and Forli,⁵ may also help to define choices among different sequential steps in a non-targeted synthesis even more concisely. Information specified by the structure of one member from a group of isomeric molecules can be considered high. The goal of non-targeted synthesis is to minimize the number of atoms while maximizing the number of possible isomers. The synthetic challenge is to prepare selectively one isomer when many others are possible. Crucially for applications in the quest for bioactive compounds, a collection of such isomers, or closely related molecules, can become a powerful tool for probing the biology if paired with suitable assay for biological activity.⁶

### SUMMARY

In an aspect, this disclosure provides a compound of Formula I, Formula II, or Formula III

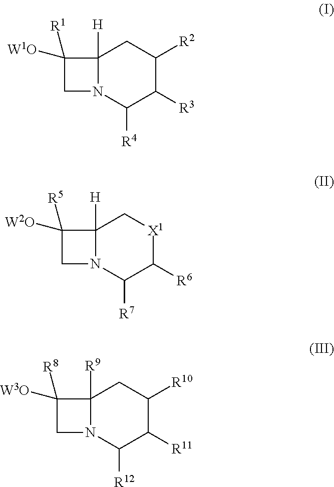

or a pharmaceutically acceptable salt and/or solvate thereof; wherein
   R¹ is aryl or heteroaryl;
   R², R³, and R⁴ are each independently H, alkyl, or halo;
   R⁵ is aryl or heteroaryl;
   X¹ is O, NH, or S;
   R⁶ and R⁷ are each independently H, alkyl, or halo;
   R⁸ is aryl or heteroaryl;
   R⁹ is alkyl;
   R¹⁰, R¹¹, and R¹² are each independently H, alkyl, or halo; and
   W¹, W², and W³ are independently at each occurrence H or

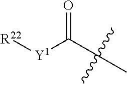

wherein Y¹ is CH₂ or CN₂, and R²² is alkyl, aryl, or heteroaryl.

In another aspect, this disclosure provides a compound of Formula IV

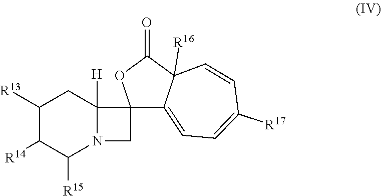

or a pharmaceutically acceptable salt and/or solvate thereof; wherein
   R¹³, R¹⁴, and R¹⁵ are each independently H, alkyl, or halo;
   R¹⁶ is aryl or heteroaryl; and
   R¹⁷ is H, alkyl, or halo.

In another aspect, this disclosure provides a compound of Formula V

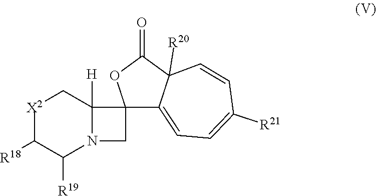

or a pharmaceutically acceptable salt and/or solvate thereof; wherein
   X² is O, NH, or S;
   R¹⁸ and R¹⁹ are each H, alkyl, or halo;
   R²⁰ is aryl or heteroaryl; and
   R²¹ is H, alkyl, or halo.

### BRIEF DESCRIPTION OF THE DRAWINGS

FIG. 1 is a graphical representation of an increase in structural complexity index (C.I.) along the reaction sequence.

FIG. 2 shows the X-ray diffraction structures of the reactant and product of Scheme 2A (carbon, gray; oxygen, red; sulfur, yellow; hydrogen, when shown, white).

FIGS. 3A-3B demonstrates that tosylate salts can also be a liability in the photochemical reaction. It was hypothesized that in the morpholine containing substrate, [18H]⁺ TsO⁻ (FIG. 3A), positioning of the tosylate parallel and proximal to the benzoyl group interferes with the subsequent y hydrogen abstraction. The arrangement of the tosylate is different in the substrate that successfully cyclizes (FIG. 3B).

FIGS. 4A-4B show ultrafast spectroscopy results of the triplet excited state, providing evidence for extended triplet state lifetime through protonation. FIG. 4A shows that the investigated Compounds 6, 18, and 20 have short half-lives, whereas their tosylate salts have long half-lives, comparable to the lifetime of the C—H analog, 1. FIG. 4B is a representation of the signal at one wavelength across 200 ps window.

FIGS. 5A-5D demonstrate that the rate of absorbed photons is directly proportional to the rate of aza-Yang reaction. FIG. 5A is an irradiance spectrum of the light source (Hg—Xe lamp) as provided by the manufacturer. FIG. 5B is the transmittance of 3 different filters used in the determination of the kinetic profile of the reaction. FIG. 5C shows the extinction coefficients of [6H]⁺ TsO⁻. FIG. 5D demonstrates that the linear relationship between Ia and reaction initial rates gives the quantum yield of the reaction from the slope of fitted lines.

FIGS. 6A-6C shows three models of the final structures. FIG. 6A shows the crystal structure of a cycloheptatriene-containing azetidine lactone hydrochloride salt (CCDC 2126589). Carbon, gray; nitrogen, blue; oxygen, red; fluorine, yellow; chlorine, green; bromine, ocher; hydrogens, white (only the ones at azetidine fusion are shown). FIG. 6B shows key HMBC correlations found for compound 1-2-1 (Scheme 5B herein) established the connectivity of the skeleton. In this scheme, the same letter is used at the origin (lower case for proton) and the terminus (upper case for carbon) when these nuclei “see” each other in HMBC experiment. Paths through three bonds (H—X—X—C) are bolded for the key HMBC relationships. FIG. 6C shows the computed model of compound 1-1-2 (Scheme 5B herein) with B3LYP DFT functional and Pople's triple split basis set with six Gaussian functions. NOE interactions between exo methylene proton on azetidine and ortho protons on the aromatic ring at distance 2.67 Å and the absence of the analogous “through-space” interaction from the methine azetidine proton suggest that the major diastereomer is the one shown. Dihedral angles in cycloheptatriene are also shown.

FIG. 7 shows the schematic route leading from acetophenones (AcAr), secondary amines (R₂NH), and arylacetic acids (ArAA) consists of seven transformations (I-VII).

FIG. 8 shows determination of bioactive treatments.

FIG. 9 shows clustering of fingerprints from cycloheptatriene collection with the known bioactives.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only and is not intended to be limiting.

Unless defined otherwise, all technical and scientific terms used herein generally have the same meaning as commonly understood by one of ordinary skill in the art to which this technology belongs.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term (e.g., except where such number would be less than 0% or exceed 100% of a possible value)—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

Generally, reference to a certain element such as hydrogen or H is meant to include all isotopes of that element. For example, if an R group is defined to include hydrogen or H, it also includes deuterium and tritium. Compounds comprising radioisotopes such as tritium, ¹⁴C, ³²P and ³⁵S are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; pentafluorosulfanyl (i.e., SFs), sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. Cycloalkenyl groups may be substituted or unsubstituted. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Cycloalkenylalkyl groups may be substituted or unsubstituted. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups may be substituted or unsubstituted. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡CH, —C≡CCH₃, —CH₂C≡CCH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Aryl groups may be substituted or unsubstituted. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. The phrase “aryl groups” includes groups containing fused rings, such as fused aromatic-aliphatic ring systems (e.g., indanyl, tetrahydronaphthyl, and the like). Representative substituted aryl groups may be mono-substituted (e.g., tolyl) or substituted more than once. For example, monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include aromatic (also referred to as heteroaryl) and non-aromatic ring compounds containing 3 or more ring members, of which one or more is a heteroatom such as, but not limited to, N, O, and S. Heterocyclyl groups may be substituted or unsubstituted. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass aromatic, partially unsaturated and saturated ring systems, such as, for example, imidazolyl, imidazolinyl and imidazolidinyl groups. The phrase “heterocyclyl group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. The phrase includes heterocyclyl groups that have other groups, such as alkyl, oxo or halo groups, bonded to one of the ring members, referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups may be substituted or unsubstituted. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, imidazopyridinyl (azabenzimidazolyl), pyrazolopyridinyl, triazolopyridinyl, benzotriazolyl, benzoxazolyl, benzothiazolyl, benzothiadiazolyl, imidazopyridinyl, isoxazolopyridinyl, thianaphthyl, purinyl, xanthinyl, adeninyl, guaninyl, quinolinyl, isoquinolinyl, tetrahydroquinolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Heterocyclylalkyl groups may be substituted or unsubstituted. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Heteroaralkyl groups may be substituted or unsubstituted. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Alkoxy groups may be substituted or unsubstituted. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸², and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to —NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

The term “pentafluorosulfanyl” refers to —SF₅.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. For example, in aqueous solution, quinazolinones may exhibit the following isomeric forms, which are referred to as tautomers of each other:

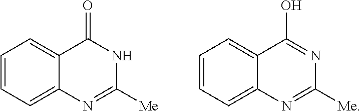 [SMILES: Cc1nc2ccccc2c(=O)[nH]1 ; Cc1nc(O)c2ccccc2n1]

As another example, guanidines may exhibit the following isomeric forms in protic organic solution, also referred to as tautomers of each other:

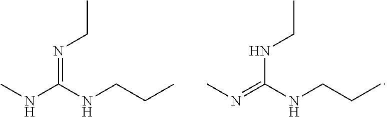

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

The compounds of the present technology may exist as solvates, especially hydrates. Hydrates may form during manufacture of the compounds or compositions comprising the compounds, or hydrates may form over time due to the hygroscopic nature of the compounds. Compounds of the present technology may exist as organic solvates as well, including DMF, ether, and alcohol solvates among others. The identification and preparation of any particular solvate is within the skill of the ordinary artisan of synthetic organic or medicinal chemistry.

Throughout this disclosure, various publications, patents, and published patent specifications are referenced by an identifying citation. Also within this disclosure are Arabic numerals referring to referenced citations, the full bibliographic details of which are provided immediately preceding the claims. The disclosures of these publications, patents, and published patent specifications are hereby incorporated by reference into the present disclosure to more fully describe the state of the art to which this invention pertains.

### The Present Technology

A collection of complex cycloheptatriene-containing azetidine lactones was prepared by applying two key photochemical reactions: “aza-Yang” cyclization and Buchner carbene insertion into aromatic rings. While photolysis of phenacyl amines leads to a rapid charge transfer and elimination, it was found that a simple protonation of the amine enables the formation of azetidinols as single diastereomers. Through ultrafast spectroscopy, evidence is provided for the electron transfer from free amines in the excited state. Further, the aza-Yang reaction was characterized by establishing the dependence of initial reaction rates on rates of photon absorption. The unexpected change in reactivity in the morpholine analogs is explained through interactions with the tosylate anion. The Buchner reaction proceeds with a slight preference for one diastereomer over the other, and successful reactions require electron-donating carbene-stabilizing substituents.

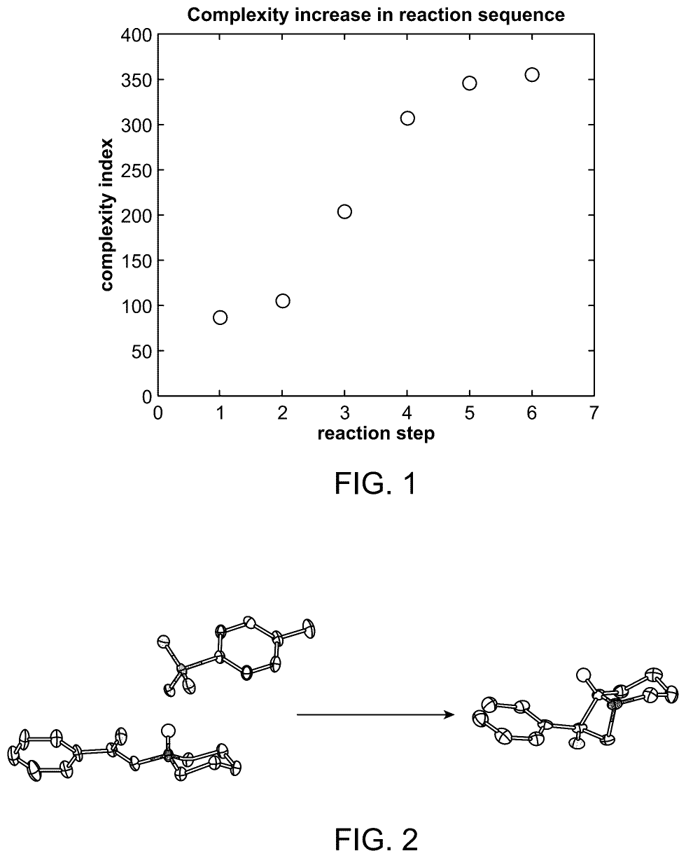

In Scheme 1, provided is a sample non-targeted synthetic strategy aimed at constructing a collection of “non-natural” complex structures occupying new chemical space. The strategy is based on two key photochemical reactions that achieve a significant and rapid increase in structural complexity, and generate appealing motifs, i.e., rigidified polycyclic, spirocyclic, or fused systems. As an example, azetidinols were first prepared through a modified Norrish-Yang cyclization named the “aza-Yang” or “aza-Yang cyclization” (FIG. 1A, aza-Yang).⁷ The tertiary alcohol obtained in the aza-Yang product was then used to install a group on which a carbene precursor could be introduced. Finally, an intramolecular Buchner reaction^{8,9} completes the sequence (FIG. 1A, Buchner). This de-aromatization reaction inserts a carbene into a benzene ring carried through from the photochemical aza-Yang reaction, contributing to the overall high utilization of functionalities present in the starting materials. The products of this reaction sequence contain three stereocenters (one of which is all-carbon quaternary), 4-, 5-, 6-, and 7-membered rings, a tertiary amine, a γ-lactone, and a cycloheptatriene; overall, a lot of new functionality for just a few synthetic steps.

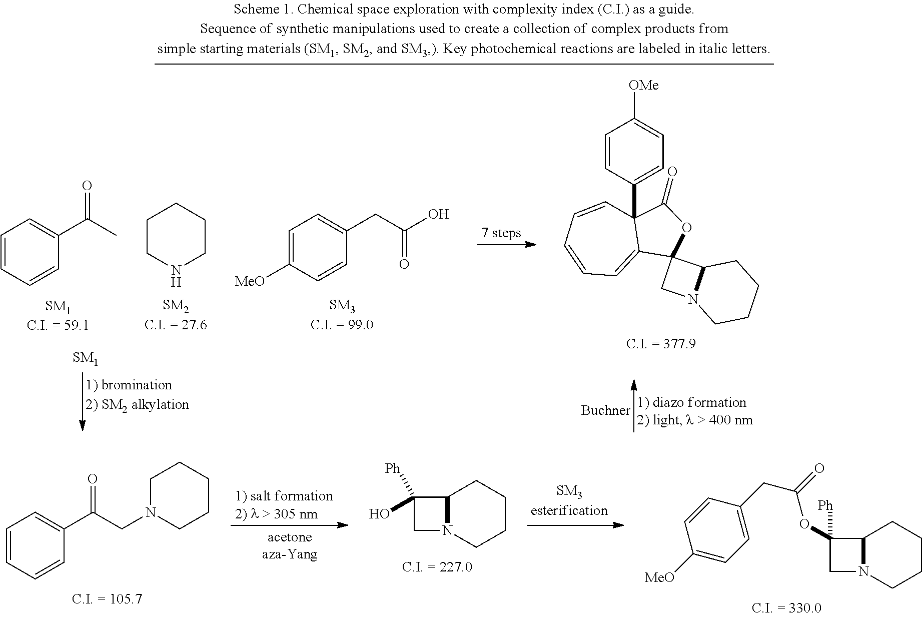

Tracking the increase in structural complexity4 across the sequence of seven reactions (FIG. 1) shows that the aza-Yang reaction doubles the complexity of the simple phenacyl-piperidine (C.I. 203.5 versus 105.7, Scheme 1). A large increase in C.I. is obtained in the coupling step, (3 to 4), but this is more due to new atoms and connectivity being introduced when two molecules are joined together. The flattening of the complexity curve suggests a natural stop to the reaction sequence. Members of the final compound collection (Scheme 5B) have a C.I. in the range from 293 to 383. For perspective, raw Böttcher complexity indices of several bioactive compounds, such as penicillin, lovastatin, vincristine, or vancomycin are listed in Table 1 along with C.I. values normalized for molecular weight.

TABLE 1
Computed Böttcher complexity indices (C.I.) of several
“famous” bioactive molecules and of one of the compounds
synthesized using the methods described herein. Complexity normalized
by molecular weight as a proxy for size of these molecules is also shown.

| name | C.I. | C.I./MW |
|---|---|---|
| penicillin | 298.95 | 0.893 |
| lovastatin | 288.00 | 0.712 |
| vincristine | 959.69 | 0.707 |
| vancomycin | 1832.34 | 1.26 |
| 1-1-1, Scheme 5B | 354.5 | 1.01 |

As mentioned, FIG. 1 provides a simple example of a sequence leading to non-targeted products, which nevertheless resemble drug-like molecules and are likely to exhibit drug-like physicochemical properties. The value of such molecules lies in their use as inputs to non-targeted screening for bioactivity. Just as in targeted synthesis, the path in FIG. 1 raises all the usual questions about reaction mechanisms, selectivity, functional group tolerance, etc. Thus, by pursuing the non-targeted approach, the described methods do not depart from the themes of synthetic chemistry but gain in accessing new chemical (and biological) space.

Thus, in an aspect, the present technology provides a compound of Formula I, Formula II, or Formula III

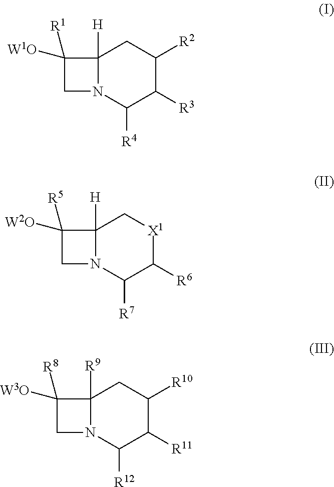

or a pharmaceutically acceptable salt and/or solvate thereof; wherein
   R¹ is aryl or heteroaryl;
   R², R³, and R⁴ are each independently H, alkyl, or halo;
   R⁵ is aryl or heteroaryl;
   X¹ is O, NH, or S;
   R⁶ and R⁷ are each independently H, alkyl, or halo;
   R⁸ is aryl or heteroaryl;
   R⁹ is alkyl;
   R¹⁰, R¹¹, and R¹² are each independently H, alkyl, or halo; and
   W¹, W², and W³ are independently at each occurrence H or

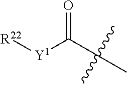

wherein Y¹ is CH₂ or CN₂, and R²² is alkyl, aryl, or heteroaryl.

In any embodiment herein, it may be R², R³, and R⁴ are each independently H, C₁-C₃ alkyl (e.g., methyl), or halo. In any embodiment herein, it may be R⁶ and R⁷ are each independently H, C₁-C₃ alkyl (e.g., methyl), or halo. In any embodiment herein, it may be R⁹ is C₁-C₃ alkyl (e.g., methyl).

In one or more embodiments, W¹, W², and W³ are independently at each occurrence H. In one or more embodiments, W¹, W², and W³ are independently at each occurrence

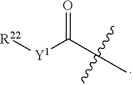

In one or more embodiments, Y¹ is CH₂. In one or more embodiments, Y¹ is CN₂.

In one or more embodiments, the compound is one of Formula IA, Formula IB, Formula IC, or Formula ID

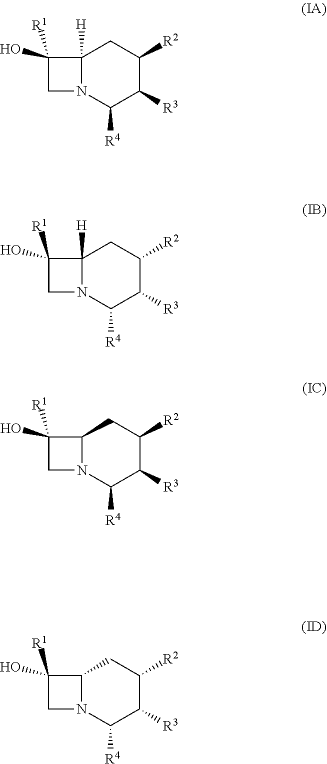

or a pharmaceutically acceptable salt and/or solvate thereof.

In one or more embodiments, the compound is one of Formula IIA, Formula IIB, Formula IIC, or Formula IID

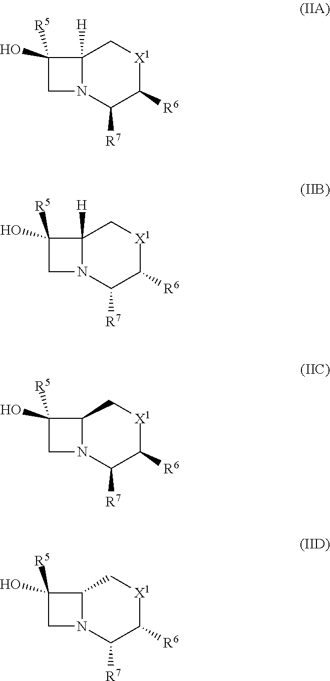

or a pharmaceutically acceptable salt and/or solvate thereof.

In one or more embodiments, the compound is one of Formula IIIA or Formula IIIB

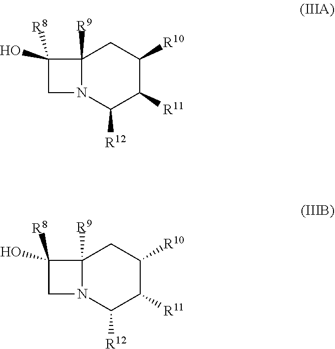

or a pharmaceutically acceptable salt and/or solvate thereof.

In one or more embodiments, the compound is one of Formula VIA, Formula VIB, Formula VIC, or Formula VID

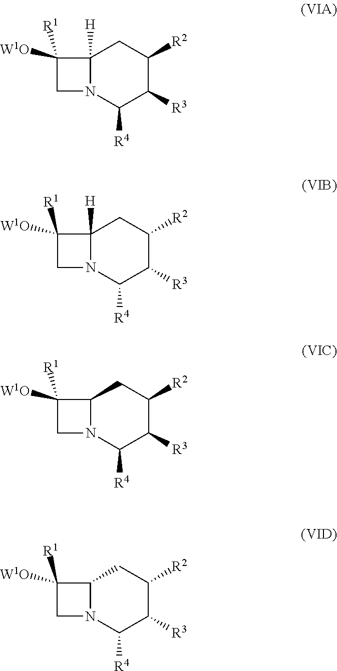

or a pharmaceutically acceptable salt and/or solvate thereof.

In one or more embodiments, the compound is one of Formula VIIA, Formula VIIB, Formula VIIC, or Formula VIID

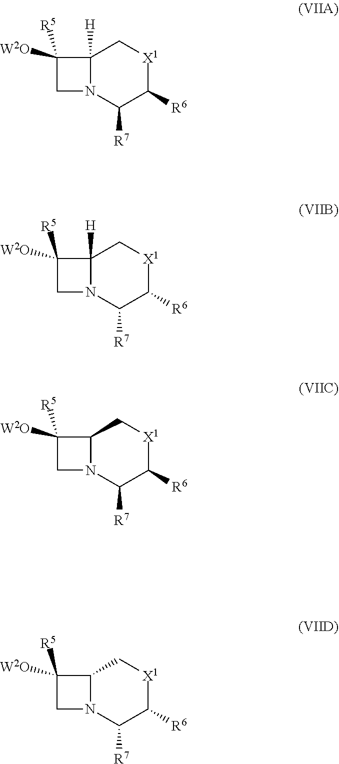

or a pharmaceutically acceptable salt and/or solvate thereof.

In one or more embodiments, the compound is one of Formula VIIIA or Formula VIIIB

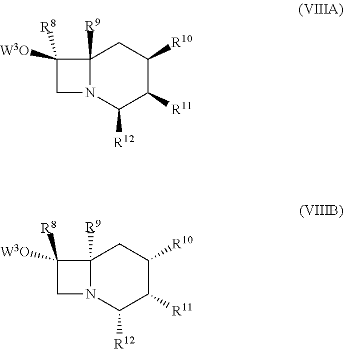

or a pharmaceutically acceptable salt and/or solvate thereof.

In another aspect, the present technology provides a compound of Formula IV

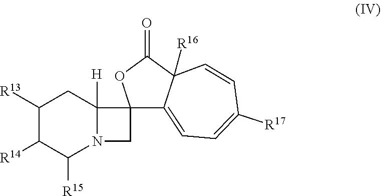

or a pharmaceutically acceptable salt and/or solvate thereof; wherein
   R¹³, R¹⁴, and R¹⁵ are each independently H, alkyl, or halo;
   R¹⁶ is aryl or heteroaryl; and
   R¹⁷ is H, alkyl, or halo.
      In any embodiment herein, it may be R¹³, R¹⁴, and R¹⁵ are each independently H, C₁-C₃ alkyl (e.g., methyl), or halo. In any embodiment herein, it may be R¹⁶ is a substituted phenyl, an unsubstituted phenyl, a substituted thiophenyl, an unsubstituted thiophenyl, or a nitrogen-containing heteroaryl. In any embodiment herein, it may be R¹⁷ is H, C₁-C₃ alkyl (e.g., methyl), or halo.

In any embodiment herein, the compound may be one of Formula IVA or Formula IVB

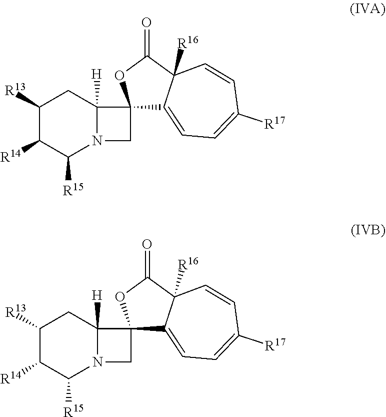

or a pharmaceutically acceptable salt and/or solvate thereof.

In an aspect, the present technology provides a compound of Formula V

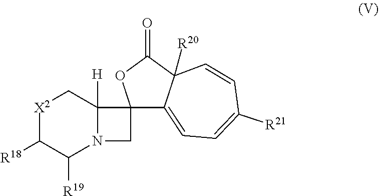

or a pharmaceutically acceptable salt and/or solvate thereof; wherein
   X² is O, NH, or S;
   R¹⁸ and R¹⁹ are each H, alkyl, or halo;
   R²⁰ is aryl or heteroaryl; and
   R²¹ is H, alkyl, or halo.
      In any embodiment herein, it may be R¹⁸ and R¹⁹ are each independently H, C₁-C₃ alkyl (e.g., methyl), or halo. In any embodiment herein, it may be R²⁰ is a substituted phenyl, an unsubstituted phenyl, a substituted thiophenyl, an unsubstituted thiophenyl, or a nitrogen-containing heteroaryl. In any embodiment herein, it may be R²¹ is H, C₁-C₃ alkyl (e.g., methyl), or halo.

In any embodiment herein, the compound may be one of Formula VA or Formula VB

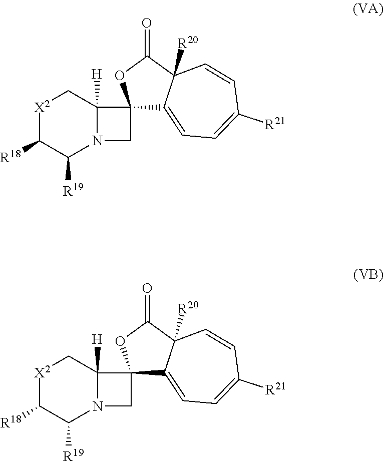

or a pharmaceutically acceptable salt and/or solvate thereof.

In any aspect or embodiment herein, the compound may be a compound as provided in Table 2 or pharmaceutically acceptable salt and/or solvate thereof.

TABLE 2

| Compound | Structure |
|---|---|
| XK00006728-001 | 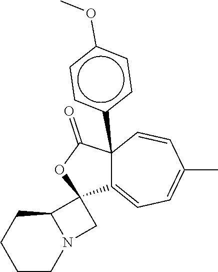 |
| XK00006719-001 | 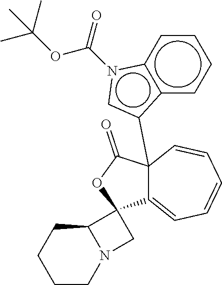 |
| XK00006729-001 | 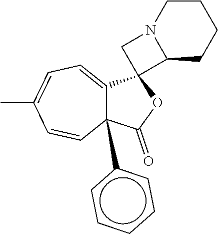 |
| XK00006721-001 | 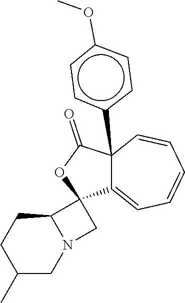 |
| XK00006730-001 | 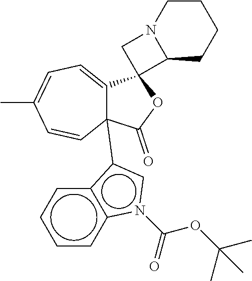 |
| XK00006722-001 | 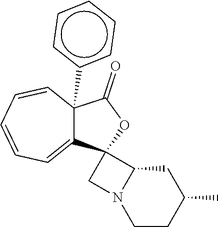 |
| XK00006731-001 | 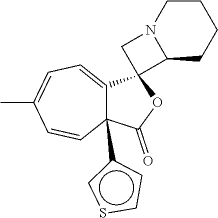 |
| XK00006723-001 | 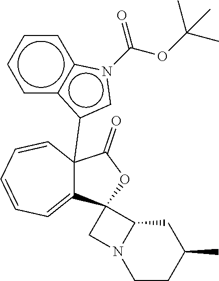 |
| XK00006726-002 | 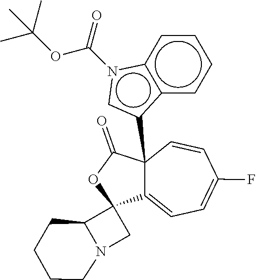 |
| XK00006724-001 | 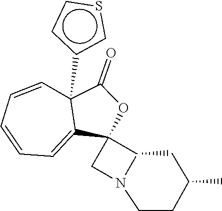 |
| XK00006720-002 | 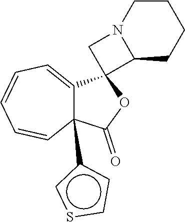 |
| XK00006716-001 | 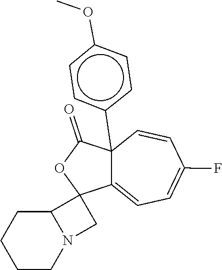 |
| XK00005456-001 | 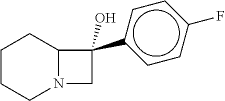 |
| XK00006725-001 | 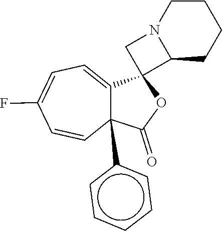 |
| XK00005530-001 | 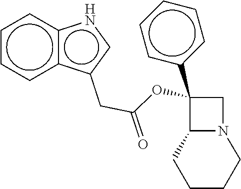 |
| XK00005533-001 | 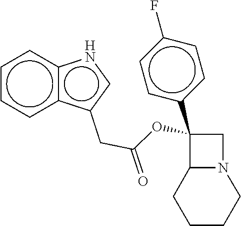 |
| XK00005498-001 | 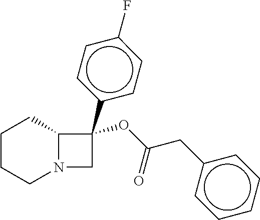 |
| XK00005531-001 | 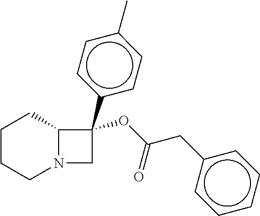 |
| XK00005532-001 | 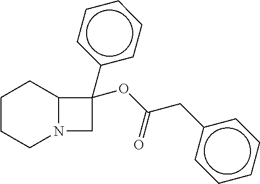 |
| XK00005472-001 | 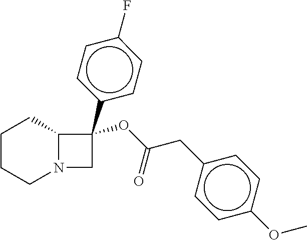 |
| XK00005468-001 | 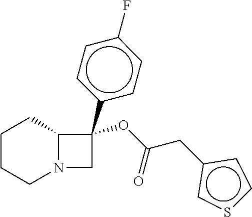 |
| XK00005306-001 | 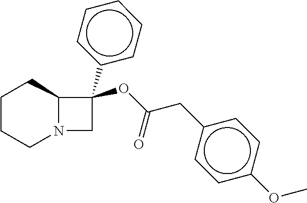 |
| XK00005525-001 | 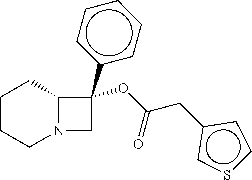 |
| XK00005471-001 | 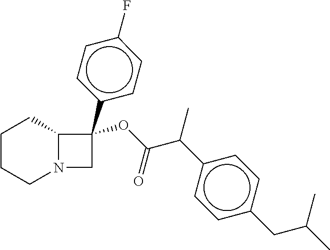 |
| XK00005495-001 | 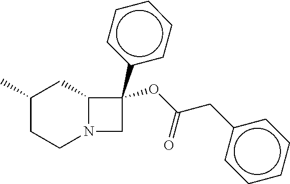 |
| XK00006712-001 | 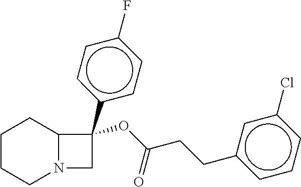 |
| XK00005454-001 | 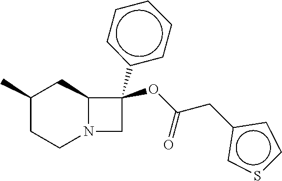 |
| XK00006714-001 | 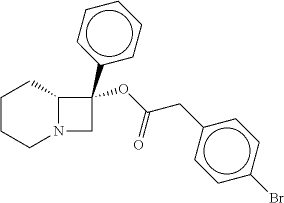 |
| XK00005489-001 | 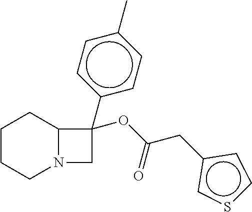 |
| XK00006713-001 | 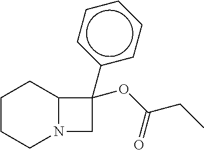 |
| XK00005436-001 | 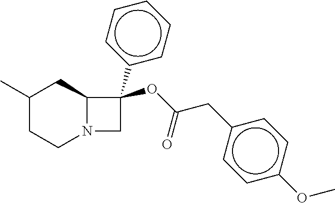 |
| XK00006715-001 | 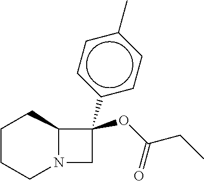 |
| XK00005488-001 | 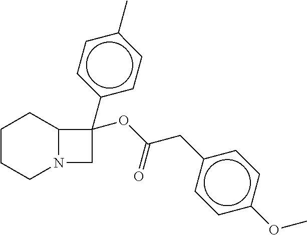 |
| XK00005493-001 | 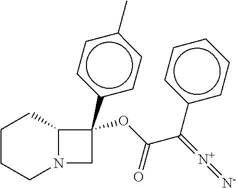 |
| XK00005474-001 | 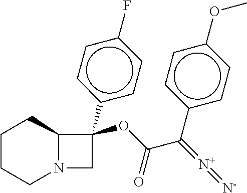 |
| XK00005439-001 | 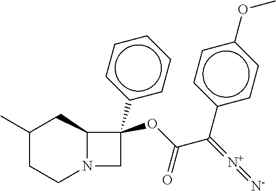 |
| XK00005496-001 | 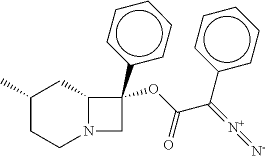 |
| XK00005534-001 | 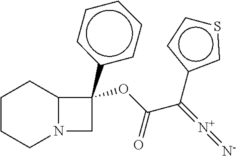 |
| XK00005492-001 | 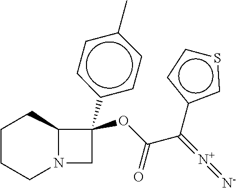 |
| XK00005522-001 | 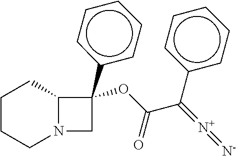 |
| XK00005494-001 | 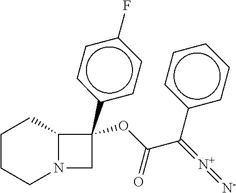 |
| XK00005486-001 | 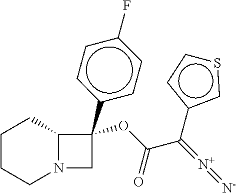 |
| XK00005497-001 | 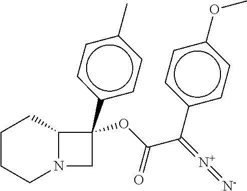 |
| XK00005450-001 | 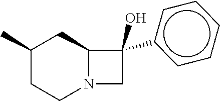 |
| XK00005466-001 | 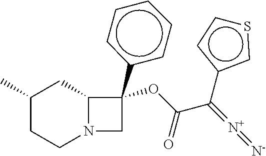 |
| XK00005490-001 | 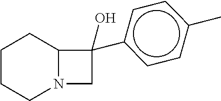 |
| XK00005311-001 | 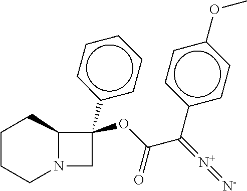 |
| XK00005441-001 | 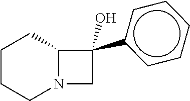 |
| XK00005440-001 | 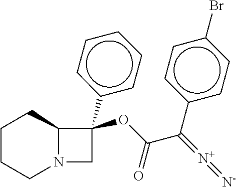 |

The examples herein are provided to illustrate advantages of the present technology and to further assist a person of ordinary skill in the art with preparing or using the compounds and compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects, or embodiments of the present technology.

### EXAMPLES

The chemistry of excited states can provide a useful entry to new structural chemical space, but challenges in controlling the highly reactive intermediates often need to be addressed. Norrish-Yang reactions produce strained, four-membered rings by converting the energy of absorbed photons to chemical energy through the interconversion of favorably oriented bonds.^{10,11} First, a carbonyl group (often an aryl ketone) absorbs a photon which allows an electron to undergo a π*←n transition. This is followed by a symmetry-forbidden singlet-to-triplet intersystem crossing. The triplet is sufficiently long lived to abstract a hydrogen radical from the γ carbon forming two carbon-centered radicals, one of them stabilized by the neighboring oxygen and the aromatic ring in the case of aryl ketones. Radical recombination of this intermediate forms the 4-membered ring.

To be experimentally acquainted with a standard Norrish-Yang cyclization, an acetonitrile solution of cyclohexyl acetophenone, Compound 1, was irradiated with a point-source Hg—Xe arc lamp.¹² A mixture of diastereomeric trans-fused cyclobutanols, Compounds 2 and 3 was obtained (Scheme 2A) as determined by X-ray diffraction of their p-bromobenzoic acid esters, Compounds 4 and 5 (CCDC 2040104 and 2039100).

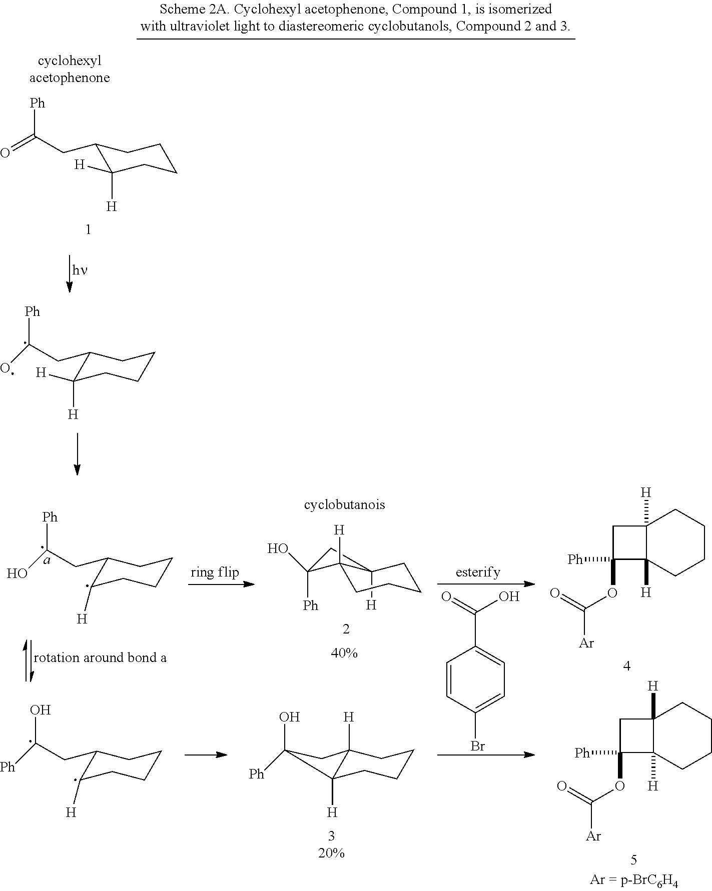

A particularly useful variant of Norrish-Yang reaction includes a nitrogen atom at the β position of the photo-excitable ketone (such as Compound 6 in Scheme 2B) and provides a route to azetidines in a simple manner. Azetidines have elicited recent interest as useful scaffolds for biologically active molecules.¹³⁻¹⁵ This approach fails, however, because of an increased electron density at the β position (a lone pair of electrons on nitrogen). More facile than γ H-radical abstraction is quenching of the triplet excited state through a rapid electron transfer from nitrogen to form a ketyl radical, which opens up a more favorable degradation pathway through the scission of the C—N bond, yielding methylketones and imine oligomers, or amines through complex reductive processes (Scheme 2B). One way to avoid this has been to protect the amine so that its lone pair engages in conjugation with other groups (e.g., by converting it to amides, carbamates, sulfonamides; Scheme 2C),¹⁶⁻¹⁸ but this necessitates additional steps (introduction and removal of the electron-withdrawing group) and precludes the use of tertiary amines.

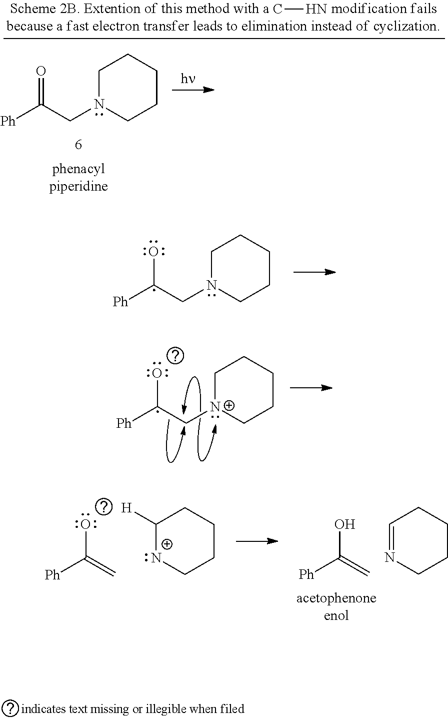

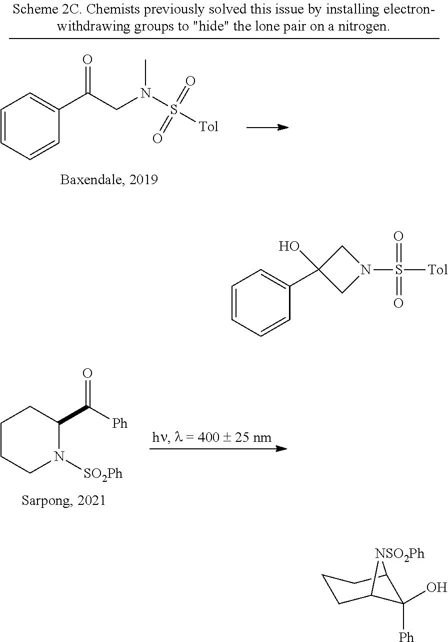

The solution presented here is to conceal the lone pair through simple salt formation (Scheme 3A). Thus, the tosylate salt of symmetric Compound 6 dissolved in acetonitrile or acetone and irradiated at wavelengths that promote π*←n transition, produces racemic Compound 7 (CCDC 2039099) as a single diastereomer with some elimination analogous to the one shown in Scheme 2B. The tosylate anion which occupies one of the faces of this molecule (top face in the crystal structure shown in FIG. 2, CCDC 2051429) may be preventing the rotation around C—C bond which in cyclohexyl acetophenone, Compound 1, leads to diastereomeric mixture of products (Scheme 2A). Larger phenyl group (A-value=3)¹⁹ stays away from the anion, which places smaller hydroxy group (A-value=0.87)¹⁹ trans from the bridgehead hydrogen. Interestingly, the final deprotonated amine has its lone pair cis with the bridgehead hydrogen, which is consistent with a Walden inversion of the nitrogen upon deprotonation. Transanular delocalization of the nitrogen's lone pair into antibonding C—O orbital (n←σ interaction) may provide sufficient driving force for this inversion.

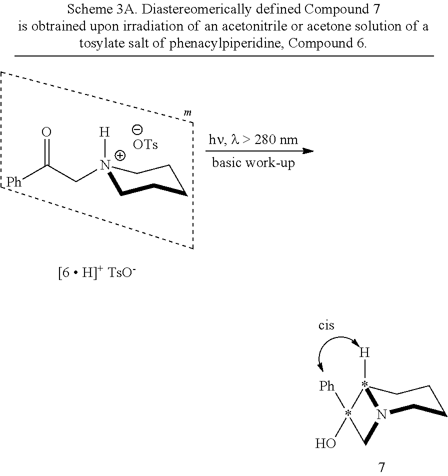

Following this protocol, azetidinols 7-17 and 19 (Scheme 3B) were prepared. Of these, Compounds 7-10 were used for further chemical elaboration into a compound collection. Compound 8 was obtained from a symmetric 4-methylpiperidine-containing substrate. The stereochemistry of the methyl substituent was established as endo (CCDC 2105274). Compounds 14 and 15 were both obtained from 3-methylpiperidine and co-crystallized as exo heterodimers (CCDC 2101761). Compounds 12 (CCDC 2126174) and 13 come from 2-methylpiperidine and suggest that the tertiary radical is favored for this cyclization notwithstanding an increase in steric congestion.

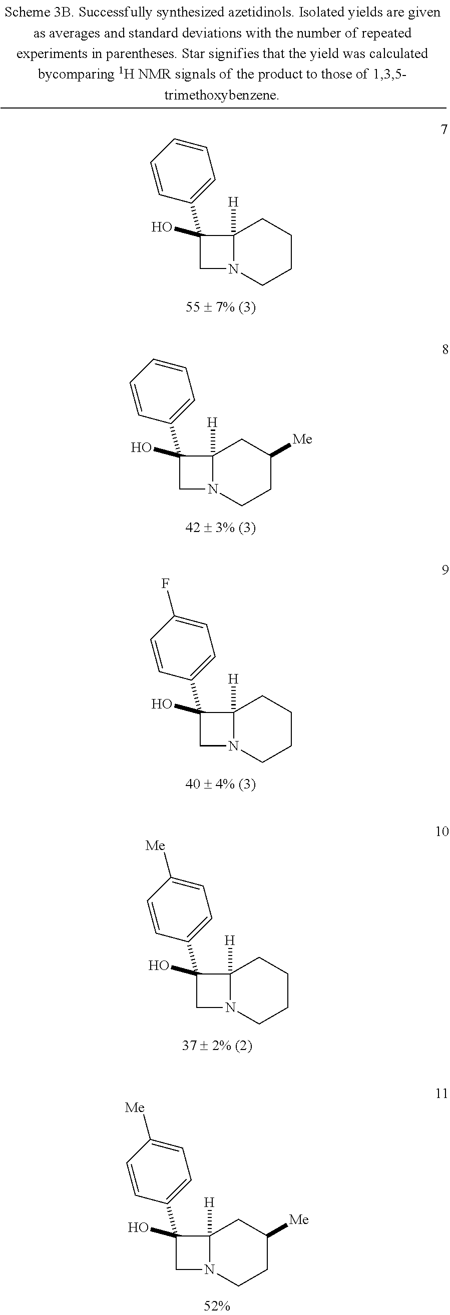 [SMILES: O[C@]1(c2ccccc2)CN2CCCC[C@@H]21 ; C[C@H]1CCN2C[C@](O)(c3ccccc3)[C@H]2C1 ; O[C@]1(c2ccc(F)cc2)CN2CCCC[C@@H]21 ; Cc1ccc([C@@]2(O)CN3CCCC[C@@H]32)cc1 ; Cc1ccc([C@@]2(O)CN3CC[C@H](C)C[C@@H]32)cc1]

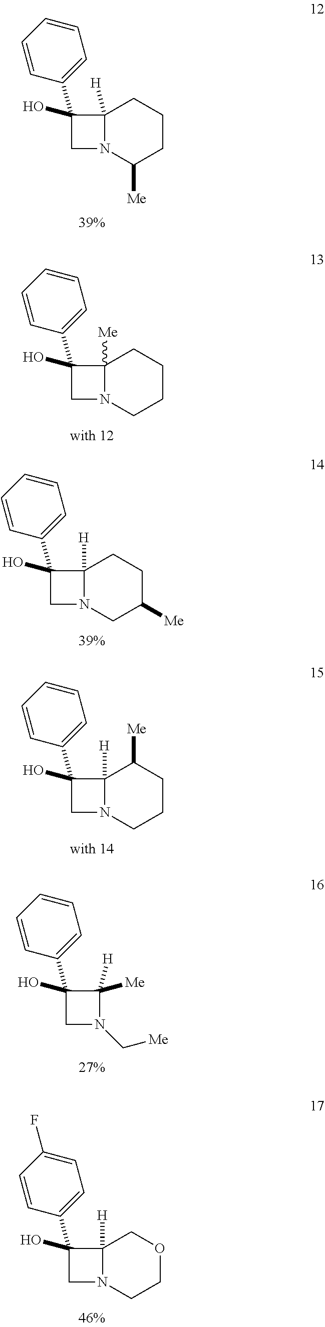 [SMILES: C[C@@H]1CCC[C@H]2N1C[C@]2(O)c1ccccc1 ; CC12CCCCN1C[C@]2(O)c1ccccc1 ; C[C@@H]1CC[C@H]2N(C1)C[C@]2(O)c1ccccc1 ; C[C@H]1CCCN2C[C@](O)(c3ccccc3)[C@@H]12 ; CCN1C[C@](O)(c2ccccc2)[C@H]1C ; O[C@]1(c2ccc(F)cc2)CN2CCOC[C@@H]21]

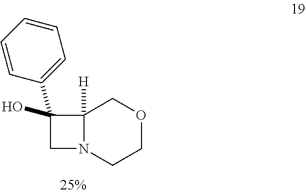 [SMILES: O[C@]1(c2ccccc2)CN2CCOC[C@@H]21]

Substrates that resisted azetidinol formation were also encountered (Scheme 3C) by either being inert or by degrading to complicated mixtures of products. For example, electron-rich substituents on the arylketones are known to preferentially undergo π*←π transition and are inert in the Norrish-Yang reaction. Acetone derivatives are not suitable substrates for this reaction, nor are substrates where the geometry prevents hydrogen radical abstraction (such as in pyrrolidines or ortho-substituted acetophenones).²⁰ If a substrate contains an aryl bromide, ultraviolet light generates aryl and bromine radicals which lead to complex mixtures of products.

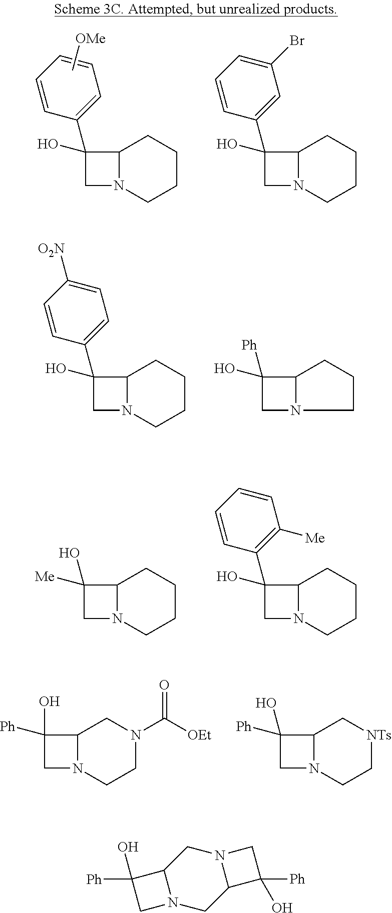

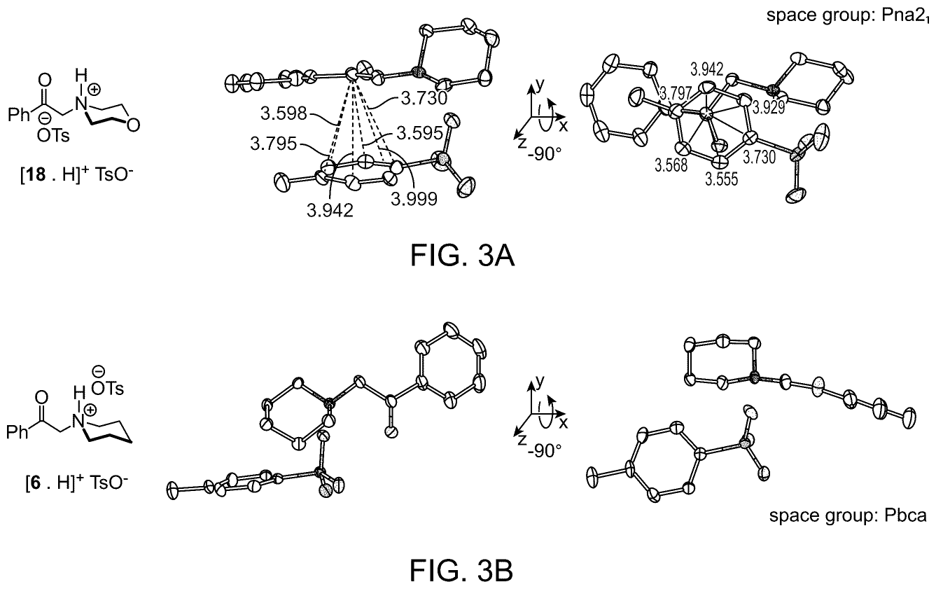

In some amines, even salt formation (through nitrogen protonation) prior to photolysis does not allow azetidine formation. For example, simple change of the substrate from phenacyl-piperidine, Compound 6, to phenacylmorpholine, Compound 18, (FIG. 3A) yielded neither the azetidinol nor the elimination products. This abrupt change in reactivity was surprising emanating from a somewhat remote oxygen in the ring. Subtle geometry changes in the substrate could account for this total lack of reactivity. Surprisingly, the solid-state structures of the salts of these two analogs (FIGS. 3A-3B) revealed a stark difference in the positioning of the tosylate anion, which may explain the origin of this shift in reactivity. In Compound 6 (CCDC 2051429), the tosylate is positioned “over” the saturated heterocycle and away from the benzoyl group, whereas in Compound 18 (CCDC 2104641), the tosylate is hovering directly over the benzoyl group that is excited. In this configuration, the proximal tosylate, at a distance of 3 Å, is ideally positioned to accept the excess energy stored in the carbonyl excited state, thereby quenching it.

To test this hypothesis, a mesylate salt of the phenacyl morpholine was prepared. Indeed, the mesylate salt engaged in photochemical reactions and produced the expected azetidinol Compound 19 (fluoro analog Compound 17 CCDC 2127602). Other acids, such as perchloric acid, acetic acid, formic acid, oxalic acid, and camphorsulfonic acid, were also tested, but tosylate salts were most ideal either for the outcomes (minimizing the elimination and maximizing cyclization) or for the ease of manipulation.

### Characterization of the Aza-Yang Triplet Excited State

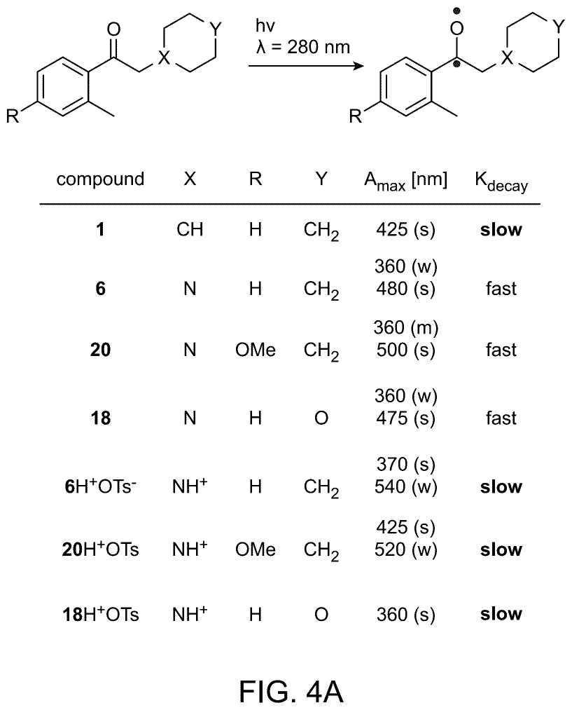

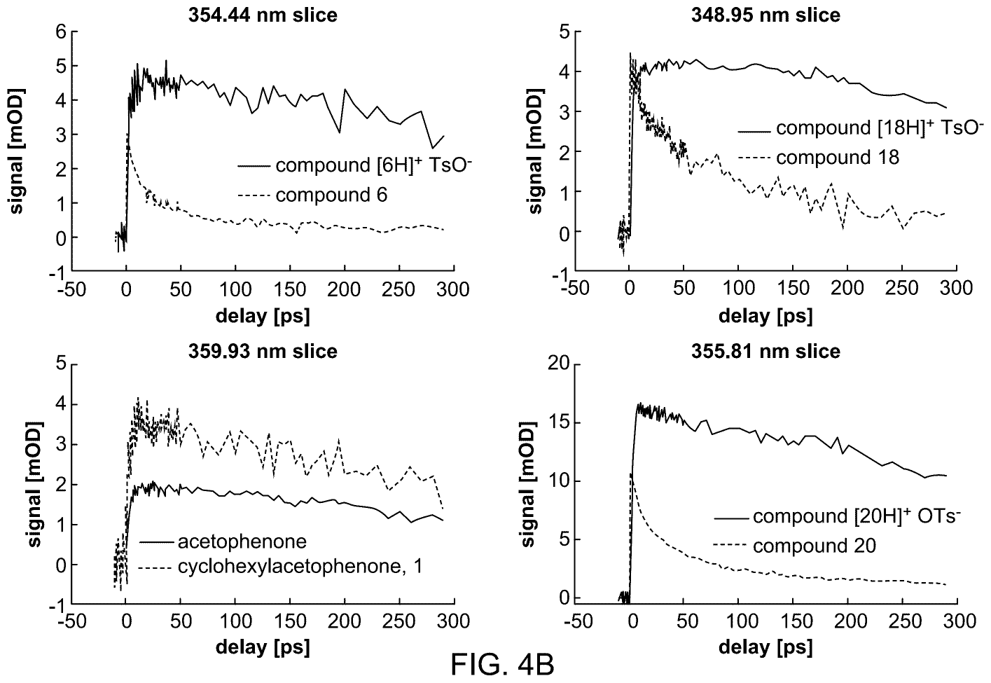

Having empirically observed that free amines prefer elimination to cyclization, whereas protonated amines cyclize to azetidinols, the differences in primary photochemical processes were investigated to determine why protonation works. Ultrafast spectroscopy was used to reveal what happens to the triplet excited state very soon after the absorption of a photon. Solutions of seven compounds (FIG. 4A) were subjected to a rapid burst of laser monochromatic light at 280 nm (corresponding to the π*←n excitation of the aryl ketone). After an ultrashort and precisely measured delay following the flash of the exciting light (from 1 ps to several hundred picoseconds), a probing beam interrogates the absorption of the species formed in the wavelength range from 334 nm to 684 nm. The spectrum of the excited state species obtained within 2 ps is the triplet excited state with a characteristic absorption at 354 nm.

This excited state of Norrish-Yang substrates in which the amines are free is very short lived. Analyses indicate that the excited state decays with two exponential decays: one with a 9 ps lifetime and another with 59 ps lifetime in the case of Compound 6. In contrast to the quick dissipation of the excited states in free amines, the excited state of protonated amines is much longer (i.e., several nanoseconds at least, beyond the range of the equipment used herein to measure) This behavior is similar to the excited state of the CH analog, cyclohexyl acetophenone Compound 1, which also has a long-lived excited state (around 100 ns was the lifetime reported for similar substrates)²¹ and yields cyclobutanols on exposure to UV light. This is clear evidence that the electron transfer from the amine to carbonyl forming ketyl radical anion and an aminium radical is responsible for the quick disappearance of the excited states of Compounds 6, 18, and 20. The product of this electron transfer is then poised for homolytic cleavage of the carbon-nitrogen bond and elimination of acetophenone enol following proton abstraction and formation of tetrahydropyridine (Scheme 2B).

### Determination of the Quantum Yield

The rate of a photochemical reaction depends on the characteristics of the light source (intensity, wavelengths), and the ability of a photo-substrate to absorb photons, in addition to the concentration of the reactants and temperature. Most commonly, the rate of the reaction is related to the rate of the absorption of photons in the reactor, Iₐ through the expression

$$- \frac{d [ S ]}{dt} = β {I}_{a}^{n} ,$$

where [S] is the substrate concentration.²² Both the factor, β, and the exponent, n, are unknown, similar to the normal treatment of non-photochemical reaction. However, the rate of photon absorption varies along the length of the reactor as the light is gradually diminished by the absorbing species, and cannot be easily systematically varied to solve for β and n.

The initial rates of the aza-Yang cyclization were studied under four different conditions: 1) variable concentration of the substrate, keeping the nominal power output of the light source at 300 W, and using a high pass, cut-off filter at 305 nm; 2) same as 1, but with the high pass filter at 280 nm; 3) same as 1, but with a band pass filter centered at 280 nm (with a 10 nm half-width); and 4) variable nominal power output with the concentration of the substrate constant at 200 μM and with the cut-off filter at 305 nm.

Initial bulk reaction rates were obtained under these conditions and were compared to the rate of photon absorption. For the initial rates of the aza-Yang reaction, the disappearance of the substrate was monitored with a UV-vis spectrophotometer by removing the reaction cell from the source light path every minute during the initial stages of the reaction and every 10 minutes later on. These measurements stopped the clock on the reaction progress, and it was verified that the reaction does not proceed in the dark. A factor analysis approach was undertaken to de-noise the UV spectra obtained,²³ and with the aid of calibration standards, the concentration of the substrate was inferred at different time points. Denoising was achieved by transforming the data through a fast Fourier transform and removing the high and low frequency terms which correspond to noise and background of measurements.²⁴ To estimate the concentrations, the most optimal calibration matrix consisted of the substrate as a salt and p-toluenesulfonic acid, which was interestingly a suitable proxy for monitoring the appearance of the product as the azetidinol (tosylate salt) products do not have any characteristic absorption of their own other than the tosylate anion. As a rule, the rates of the appearance of the product were somewhat lower than the rates of the disappearance of the starting material (approximately 10-20%). To estimate the rate of photon absorption, the following formula was applied:

$${I}_{a} = \frac{{\int }_{λ} {E}_{λ} λ ( 1 - {10}^{- {e}_{λ} bC )} d λ}{{N}_{A} Vhc}$$

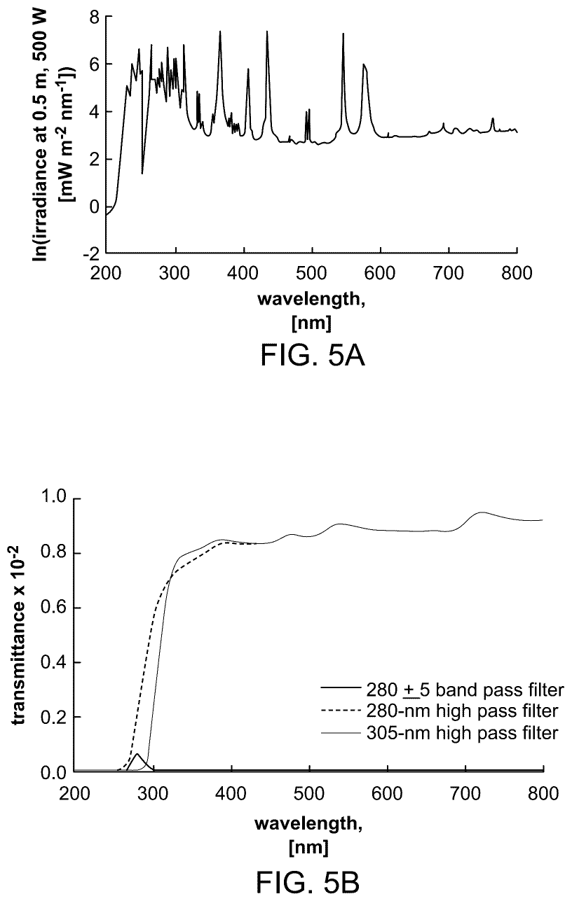

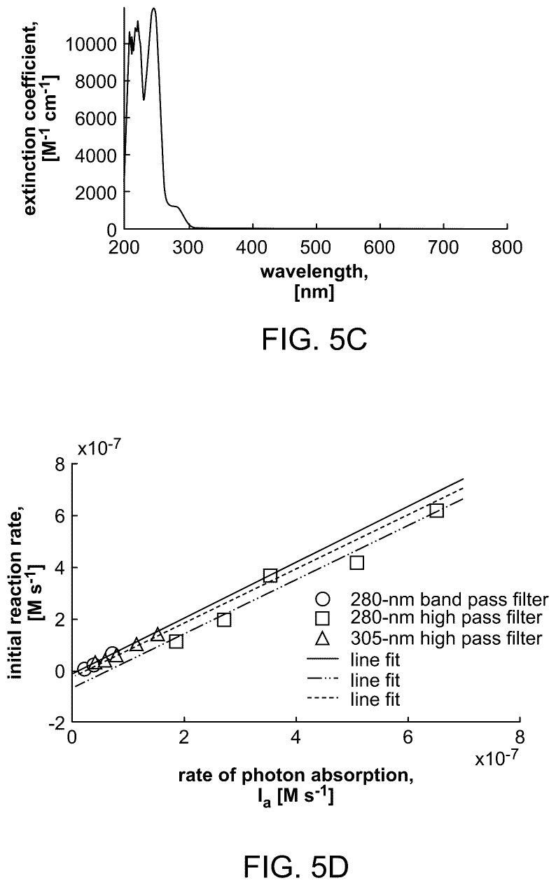

(where E_{λ} is the power of light source at wavelength λ, C is the concentration of the substrate, b is the reaction path length, NA is Avogadro's number, V is the reaction volume, h is Planck's constant, and c is the speed of light) using data on the irradiance of the light source from the manufacturer (FIG. 5A). This data shows that the power of the source varies with wavelength for a constant irradiated area at a given distance. This data needed to be corrected in two ways. First, the irradiance of the lamp is measured at 0.5 m while the reaction is at a 0.3 m distance from the source. This is easily accomplished by multiplying with 0.5²/0.3²=2.78. Second, the filters used in the reaction change the irradiance. To correct for this, the transmittance of the relevant filters was recorded in the range of 200 nm to 800 nm, and irradiance at each wavelength was multiplied with the transmittance (divided by 100) at that wavelength (FIG. 5B). Finally, the substrate's absorbing characteristics needed to be accounted for, so the data was integrated on the substrate's extinction coefficients (from 200 nm to 800 nm, FIG. 5C) with the lamp irradiance data. When the constants are included (Avogadro's number, Planck's constant, speed of light, and reaction cell volume), the rate of the absorption of photons, Iₐ, is obtained. Plotting these values for the rate of photon absorption against the initial rates obtained with different filter and substrate concentrations gives a linear dependence (FIG. 5D)

Because the dependence of initial rate on the rate of the absorbed photons follows linear and not exponential law

$$( in - \frac{d [ S ]}{dt} = β {I}_{a}^{n} , n = 1 ,$$

where [S] is the substrate concentration), the ratio of local and measured rates approaches unity. This is likely due to low absorptivity for π*←n transition. The three sets of experiments (280 nm band pass, 280 nm high pass, and 305 nm high pass filters) all give similar slopes 1.078, 1.039, and 1.036, which corresponds to the quantum yield Φ for the consumption of the substrate. Even though the absorption of the photons may be inefficient, it appears that once the photon is absorbed, the consumption of the substrate ensues.

To benchmark the rate of photon absorption against the total photon output of the light source, a chemical Hatchard-Parker actinometer was employed,²⁵⁻²⁷ which is based on photoreduction of potassium ferrioxalate, K₃[Fe(C₂O₄)₃], and spectrophotometric determination of Fe(II) complexed with 1,10-phenanthroline. Ferrioxalate has total absorption in the wavelength range from 250 nm to 500 nm. Through these measurements, the rate of photons emitted from the light source was found with the filters (from the above measurements) placed in its optical path. Since the rates of absorbed photons vary from the low of 0.022 μM/s with the lowest substrate concentration and with the 280-nm band pass filter up to 0.652 μM/s with the highest concentration and with the 280-nm high pass filter, it was concluded that between 1.3% (with the most permissive filter and the lowest substrate concentration) and 120% (with the least permissive filter and the highest substrate concentration) of the available light is absorbed by the substrate.

### Buchner Reaction

Having established a reliable route to azetidinols, they can be further structurally modified by coupling various arylacetic acids to the tertiary alcohol (created in the aza-Yang step), and then pairing this moiety with the aryl group (which had been introduced into the molecule as a chromophore for the absorption of the photon by aryl ketones) through a carbene insertion reaction.

Esterifications of the tertiary alcohols proceeded smoothly (keeping DMAP catalytic to avoid rearrangement of O-acyl to N-acyl urea which is incompetent in further coupling).²⁸ Tertiary benzylic alcohols did not epimerize under the reaction conditions (cf. crystal CCDC 2122291).

Aryl acetate esters were converted to diazo compounds by treatment with p-acetamidobenzene-sulfonylazide (p-ABSA) in the presence of diazabicycloundecane (DBU).²⁹ The esters without the aryl group had to be first benzoylated at the α position and then treated with p-ABSA, which in one step converts the B-keto esters to diazo compounds with a loss of p-acetamidobenzamide. Unfortunately, these compounds were not suitable substrates for the Buchner ring expansion reaction because they underwent a faster hydride shift to give α, β-unsaturated esters.

It is interesting to note that the carbene can add over two similar positions on the aryl group (denoted with letters a and b in Scheme 4). This divergence leads to two diastereomeric norcaradienes which upon 6-electron electrocyclic opening of the cyclopropane produce two diastereomeric cycloheptatrienes. The opening of the cyclopropane needs to proceed disrotatotary outward, which implies that this is a thermal process. Modeling predicts nearly equal total energy of these diastereomers (0.12 kcal/mol) and about a 4 kcal/mol difference between norcaradienes and cycloheptatrienes.

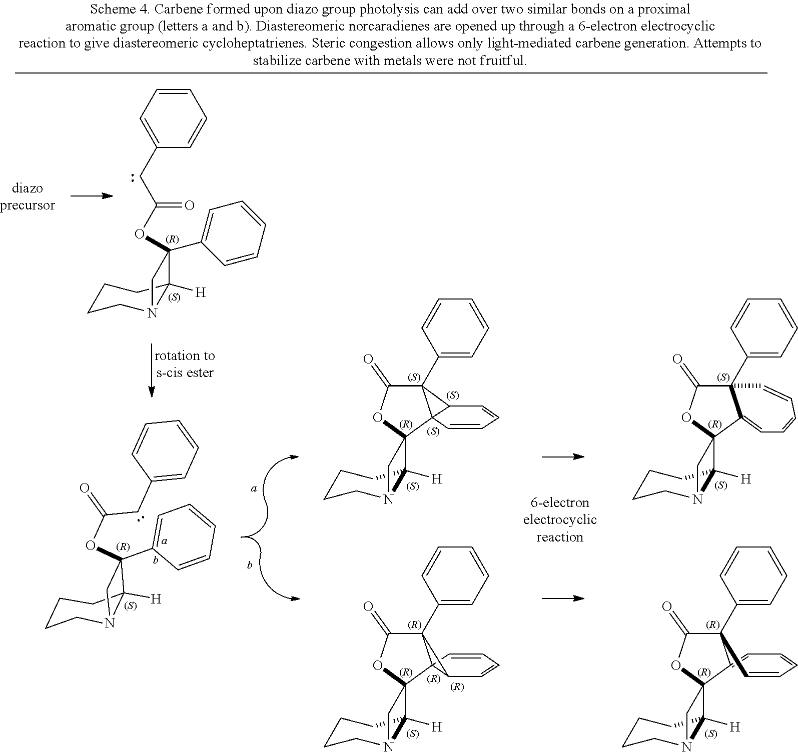

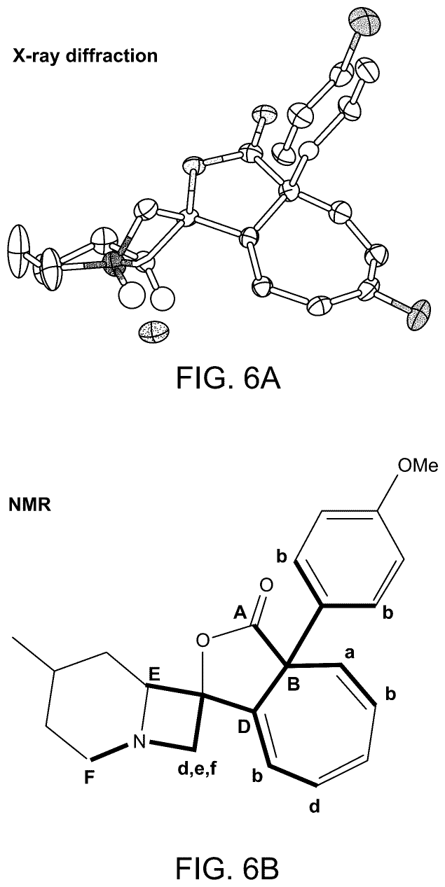

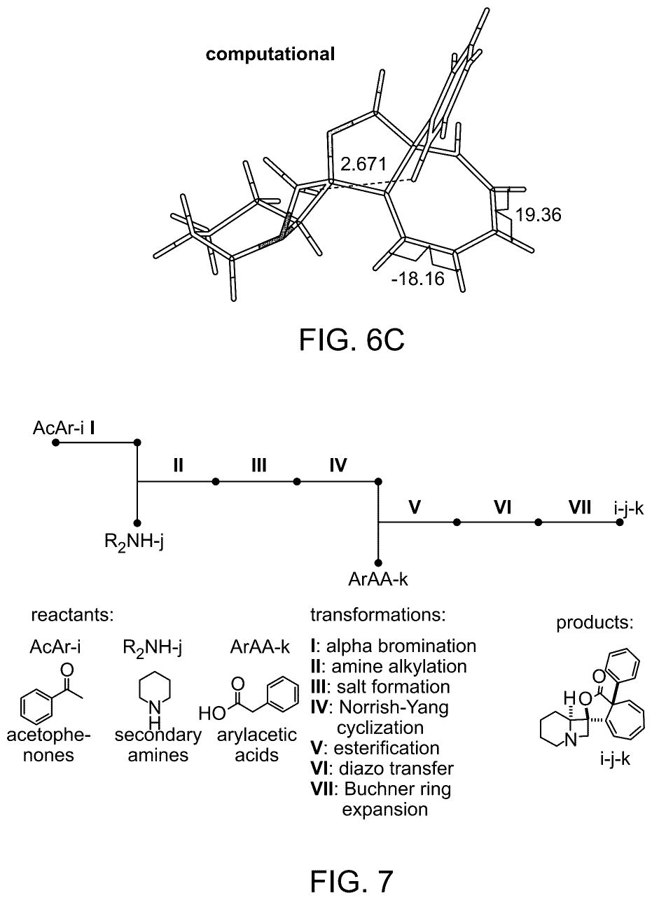

Infrared spectroscopy provided evidence for the formation of γ-lactones. Vibrational stretching frequency for γ-lactone carbonyl is 1780 cm⁻¹, whereas less strained 8-lactones vibrate at 1760 cm⁻¹. UV absorption at 270 nm is characteristic of a cyclic triene (cf. the cycloheptatriene), but this was not a diagnostic absorption as this absorption is present in the precursors as well. Stereochemistry of the all-carbon quaternary stereocenter was established through X-ray diffraction of a fluoro- and bromo-containing analog (FIG. 6A) and through NOE experiments, by observed correlation between exo methylene proton and the protons at the ortho position on the aromatic ring (FIG. 6C). Key observed HMBC correlations established connectivity for the carbon skeleton (FIG. 6A). Cycloheptatrienes are not planar and coupling constants between vinylic protons provide further evidence for its formation. In addition to coupling of protons on a cis-double bond with J₃=10 Hz, a second coupling constant of J₃=6.2 Hz is also observed, consistent with coupling in cyclic polyenes where distortion of the cycloheptatriene ring from planarity leads to dihedral angles that deviate from 0°. 30 These torsions are around 19° in computed models of both diastereomers (FIG. 6C). Karplus-Altona equation predicts 7.2 Hz coupling between protons with this torsion. 31.32 Analogous torsion from the crystal structure of a cycloheptatriene that contains a large bromine atom is 25° (FIG. 6A).

### Collection Synthesis

Ultimately, a small demonstration collection of 16 compounds was constructed based on the two key complexity-generating photochemical reactions described above. The building blocks used to make this collection are three aryl ketones, two piperidines, and four arylacetic acids (Scheme 5A). The sequence of seven transformations (FIG. 7) applied to these building blocks produces cycloheptatriene-containing azetidine lactones (Scheme 5B).

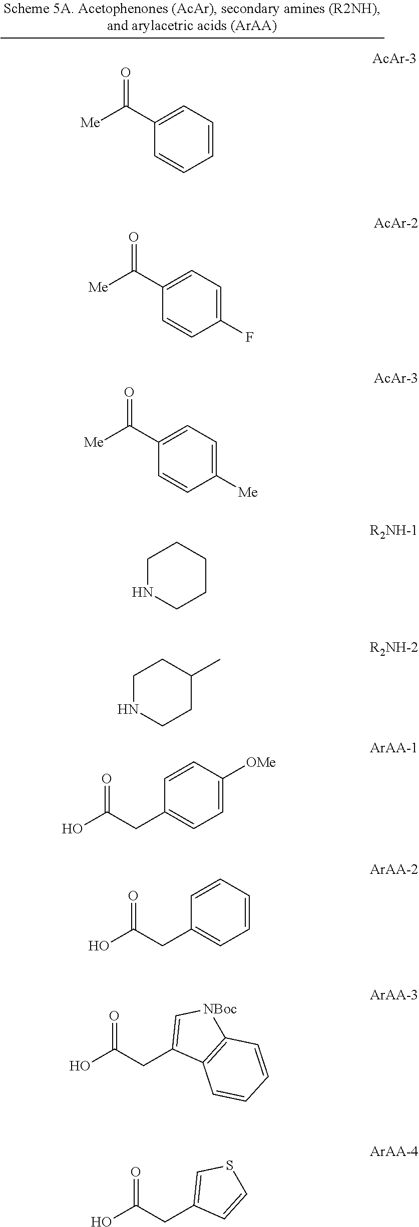

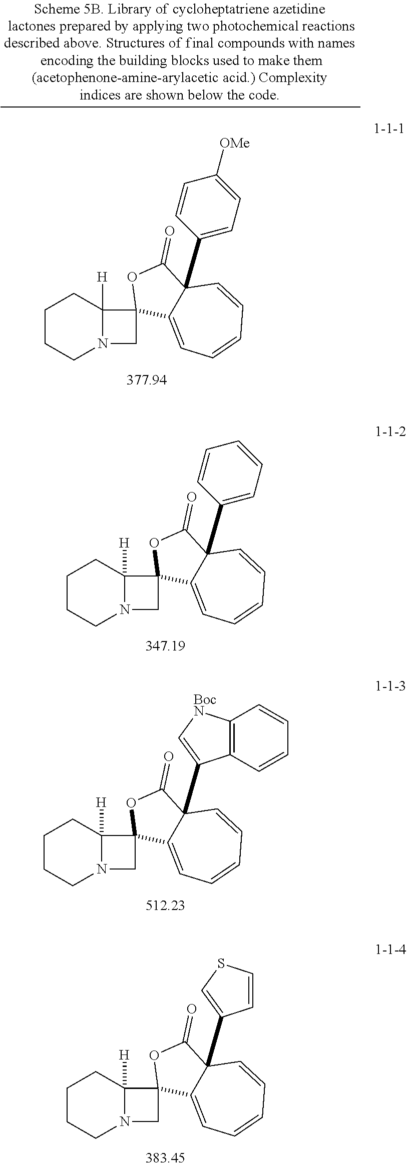

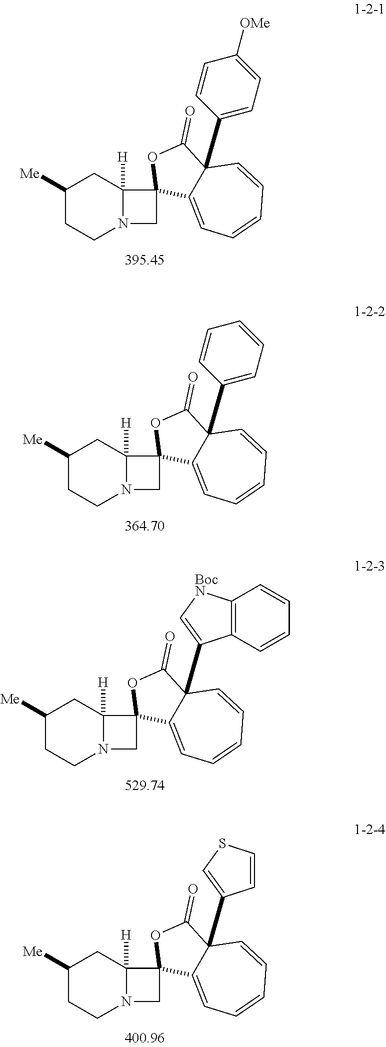

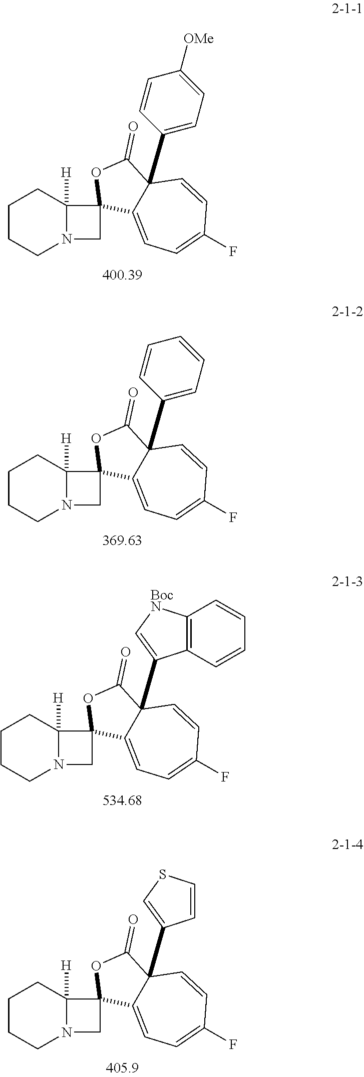

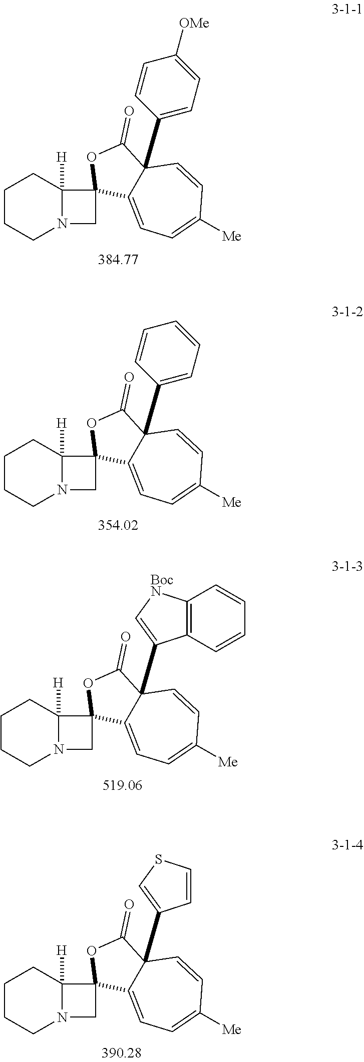

En route to compound x-y-z in Scheme 5B, the scission of the C—Br bond was observed unless the spectrum of the light source was limited to the region of blue light. A high pass filter with a cut-off at 440 nm together with a solution of CuSO₄·5H₂O (17 mM in 2.7 M ammonium hydroxide, 10 cm thickness) in front of the light beam reduced this undesired result. This is consistent with previous reports on using blue light for carbene generation.³³

### CONCLUSION

Provided is an example of how to successfully navigate to new regions of chemical space by selecting reactions that increase structural complexity along a reaction sequence. Along the way, challenges in chemoselectivity that were encountered were resolved and a firm quantitative description of the processes developed are provided.

### Example 2—Experiment Measuring Changes in Cell Morphology Induced by Synthesized Compounds

### Cell Culture

U-2 OS (ATCC, HTB-96) cells were cultured in T-75 flasks (Greiner, 658170) under McCoy's 5a medium (HIMedia, AT057), supplemented with 10% fetal bovine serum (FBS) and 1% Penicillin/Streptomycin solution, and incubated at 37° C. in a 5% CO₂ atmosphere. Upon reaching 80% confluence, cells were washed, trypsinized (0.25% solution with EDTA, VWR, M143), and removed. Flasks were reseeded with one-fifth of the cell count for each passage. All cell plates reported in this paper were composed of U-2 OS from incubation lengths of five or fewer passages. 384 Black-welled plates with optically clear bottoms (Corning, 6569) were seeded with U-2 OS at a density of 1,000 cells per well and cells were allowed to incubate for 24 h to ensure adherence.

### Compound Handling/Compound Plate

Three compound plates (Thermo Scientific, AB-1056) were constructed using an initial aliquot of a 50 mM compound solution solubilized in DMSO. Each compound aliquot was then twofold serially diluted in DMSO to yield eight concentrations ranging from 50 mM to 390 μM. This process was repeated so that compound plate 1 contained each of the 16 test (cycloheptatriene) compounds and one azetidinol, compound plate 2 contained 19 esters, and compound plate 3 contained the diazo compounds. Vehicle treatments were included in the plate design as DMSO-containing wells (around 200) with no added compound. Staurosporine diluted to 500 μM in DMSO was included on the plate as a positive control to test the transfer of compounds from pin transfer.

### Compound Treatment

After a 24 h incubation period, untreated cell-containing plates and the compound plate were each fitted into a Library Copier, and a sterile Multi-Blot Replicator (V and P Scientific) was used to transfer a 100 nL volume from each well of the 384 well compound plate into the culture media (50 μL) of a corresponding well on the 384 well cell plate. These cell plates were reintroduced to a 37° C., 5% CO₂ atmosphere for the duration of the 24-hour treatment period.

### Cell Staining/Cell Painting

### Preparation for 1 Plate

1 L of Hank's Buffered Saline Solution (HBSS) (1× from 10×) was prepared using 100 mL HBSS (10×) and diluted with 900 mL deionized water. The pH of the solution was adjusted to 7.2 with aqueous NaOH and filtered through a 0.2-micron filter. A 1% w/v solution of BSA (VWR, 0332) was prepared in 1×HBSS using 150 mg BSA, dissolved in 15 mL HBSS. The solution was filtered through a 0.2-micron filter. Live-cell MitoTracker (Thermo Fisher, M22426) staining solution was prepared using 6.5 μL of MitoTracker staining solution from a stock of 1 mM MitoTracker and dissolved in 13 mL of pre-warmed cell culture medium (RPMI) for a final concentration of 500 nM.

Staining dyes: a. Wheat Germ Agglutinin/Alexa Fluor 555 was prepared using 19.5 μL from stock solution (1 mg/mL) to make the working concentration of 1.5 ug/mL; HOECHST 33342 was prepared using 6.5 μL from stock solution (10 mg/mL) to make a working concentration of 5 μg/mL; SYTO 14 was prepared using 7.8 μL from stock solution (5 mM) to make a working concentration of 3 μM; Phalloidin/Alexa Flour 568 was prepared using 65 μL from the stock to obtain a working concentration of 5 μL/mL; and Concanvalin/Alexa Fluor 488 was prepared using 350 μL per plate (from previously prepared vials at 5 μg/mL). The volume of all staining dyes (except mitotracker), i.e, (19.5+6.5+7.8+65+350)=448.8 μL, was dissolved in 12.5 mL of 1% (wt/vol) BSA solution in HBSS.

0.1% (vol/vol) Triton X-100 (VWR, M143) was prepared using 15 μL Triton X-100+14985 μL HBSS=15000 μL. 16% (wt/vol) Paraformaldehyde (PFA, Electron Microscopy Sciences, 157110-S), methanol-free was used for fixing cells.

### Procedure for Cell Painting of One 384 Well Plate

Upon completion of the treatment period, cell plates had their media removed using a BioTek 405TS plate washer. The aspiration height in the plate washer was set in a way to leave 10 μL of residual volume in the wells in order to minimize the disturbance to the live cells from the pins and media turbulence. 30 μL of the Mitotracker (Thermo Fisher, M22426) staining solution prepared above was added to the 384-wells cell plate. The plate was centrifuged at 500 g for 1 min at room temperature and was checked to ensure there were no bubbles at the bottom of the wells. The plate was incubated for 30 min in the dark at 37° C. in 5% CO₂. The cells in the wells were fixed using 10 μL of 16% (wt/vol) methanol-free PFA (Electron Microscopy Sciences, 157110-S) for a final concentration of 3.2% (vol/vol). The plate was centrifuged at 500 g for 1 min at room temperature and was checked to ensure there were no bubbles at the bottom of the wells. The plate was incubated for 20 min in the dark at room temperature. The plate was washed with 70 μl of 1×HBSS (total volume of HBSS required=28 mL). Subsequently, HBSS was removed using the plate washer, and 30 μl of 0.1% (vol/vol) Triton X-100 solution prepared above was added to the wells. The plate was centrifuged at 500 g for 1 min at room temperature and was checked to ensure there were no bubbles at the bottom of the wells. The plate was incubated for 20 min in the dark at room temperature. The wells were washed twice with 70 μl of 1×HBSS (total volume of HBSS required=56 mL), and residual HBSS was removed. 30 μl of the phalloidin, concanavalin A, Hoechst, WGA, and SYTO 14 staining solution prepared above was added to each well. The plate was centrifuged at 500 g for 1 min at room temperature and was checked to ensure there were no bubbles at the bottom of the wells. The plate was incubated for 30 min in the dark at room temperature. The wells were washed three times with 70 μl of 1×HBSS (total volume of HBSS required (70 μL×3 times×400 well=84 mL), with no final aspiration. Finally, the plate was sealed with adhesive foil and stored at 4° C. in the dark. Experiments were repeated 3 times with cells from different passages.

### Imaging

Sealed plates were imaged using an ImageXpress Micro XL microscope and MetaXpress image acquisition software. Five wavelengths corresponding to channels, DAPI, GFP, Cy3, Cy5, and TxRed, were used to excite fluorescence and capture images of each well at 10× magnification, and with 2×2 binning. Each well was divided into four quadrants in order to capture the entire well at the selected magnification. Whole plate imaging was preceded by an initial focusing step using the DAPI channel.

### Data Analysis

Cell Profiler output comprises extracted feature values from 5 image sets for each of four sites of each of 384 wells of compound- or vehicle-treated cells. Features are initially measured per biological object (cell, nucleus, or cytoplasm) and can be averaged by Cell Profiler to give per-site values that were subsequently averaged. The average feature values were first calculated for DMSO-treated cells (190 wells on the control plate, 41 on the test plate) together with the variability in these measurements, which was represented as the standard deviation of the measurements. The DMSO-derived averages were then subtracted from the treatment conditions and this number was divided by the standard deviation to obtain the Z-scaled value for each feature. From the Z-scaled values, the magnitudes of feature vectors were computed by taking their norms, and correlations between replicates or between the active compounds (including controls) were computed by taking the inner products of the appropriate feature vectors and dividing by the products of their lengths (cosine similarity). The magnitude of feature vectors is a measure of the difference between the compound and vehicle treatments. The correlation between replicates or between the active compounds is a measure of the similarity of those conditions.

Determination of Bioactives from the Collection of Cycloheptatrienes (CHT)

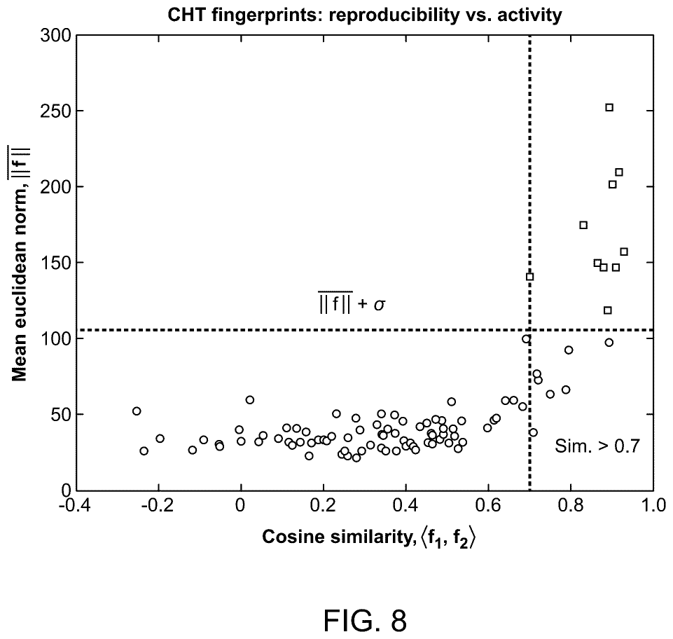

Out of the 16 final compounds synthesized in the collection of cycloheptatrienes, two compounds, indole and thiophene containing compounds 1-1-4 (Compound xk00006720) and 1-1-3 (Compound xk00006719), showed reproducible fingerprints and magnitude of the activity (FIG. 8). Further, the clustering of the compounds in this collection based on the similarity of fingerprints indicated a separate clade of these novel molecules, not overlapping with any of the compounds included in the experiment as a control.

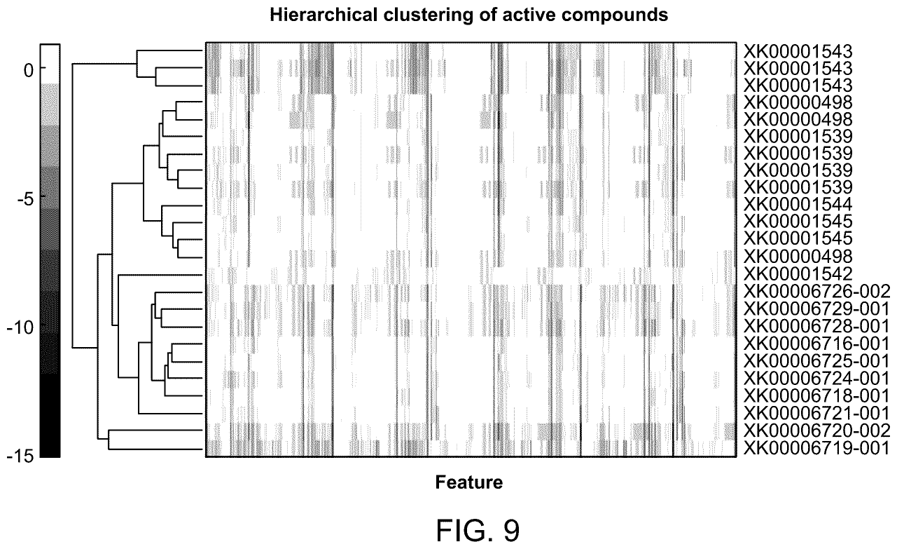

Further, it is important to note that control compounds with the same mechanism of action at different concentrations cluster together in this assay, e.g., taxol (XK00000498), and vincristine (XK00001539). In the separate cluster of the test compounds, sub-clustering of the compounds can be observed (FIG. 9), with special attention to the clade of Compound XK00006719 (1-1-3) and Compound XK00006720 (1-1-4).

TABLE 3
Compound number identification for control compounds.

| Identification | |
|---|---|
| Number | Compound |
| XK00000498 | taxol |
| XK00001539 | vincristine |
| XK00001540 | latrunculin b |
| XK00001541 | etoposide |
| XK00001542 | berberine chloride |
| XK00001543 | cytochalasin D |
| XK00001544 | fenbendazole |
| XK00001545 | rotenone |
| XK00001546 | NPPB |
| XK00001547 | SB203580 |
| XK00001548 | CA074 methyl ester |
| XK00001549 | tetrandrine |
| XK00001550 | rapamycin |
| XK00006711-001 | 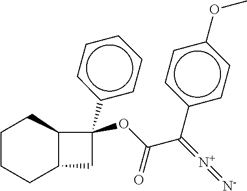 |
| XK00005313-002 | 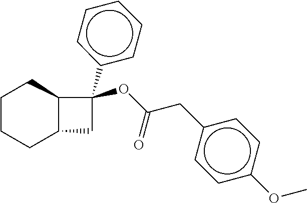 |

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A compound of Formula I, Formula II, or Formula III

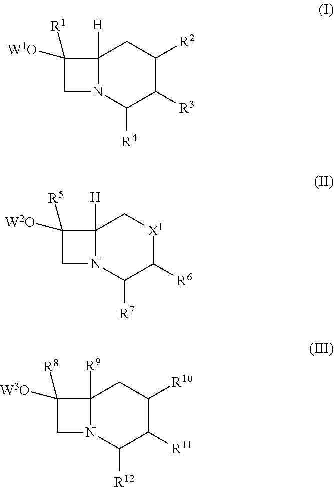

or a pharmaceutically acceptable salt and/or solvate thereof; wherein
   R¹ is aryl or heteroaryl;
   R², R³, and R⁴ are each independently H, alkyl, or halo;
   R⁵ is aryl or heteroaryl;
   X¹ is O, NH, or S;
   R⁶ and R⁷ are each independently H, alkyl, or halo;
   R⁸ is aryl or heteroaryl;
   R⁹ is alkyl;
   R¹⁰, R¹¹, and R¹² are each independently H, alkyl, or halo; and
   W¹, W², and W³ are independently at each occurrence H or

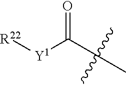

wherein Y¹ is CH₂ or CN₂, and R²² is alkyl, aryl, or heteroaryl.
B. The compound of Paragraph A, wherein W¹, W², and W³ are independently at each occurrence H.
C. The compound of Paragraph A or Paragraph B, wherein the compound is one of Formula IA, Formula IB, Formula IC, or Formula ID

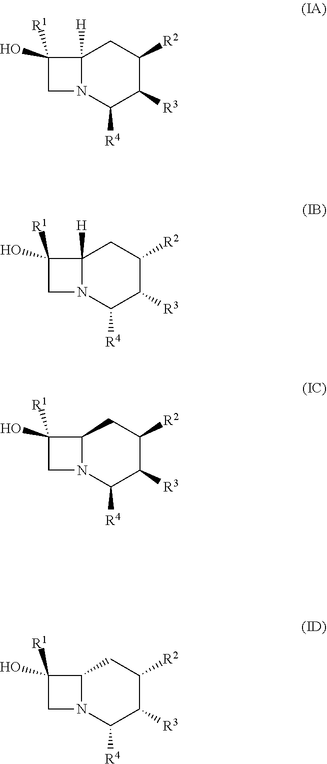

or a pharmaceutically acceptable salt and/or solvate thereof.
D. The compound of Paragraph A or Paragraph B, wherein the compound is one of Formula IIA, Formula IIB, Formula IIC, or Formula IID

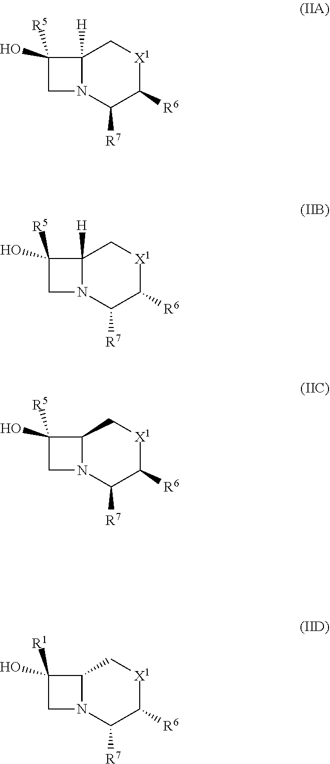

or a pharmaceutically acceptable salt and/or solvate thereof.
E. The compound of Paragraph A or Paragraph B, wherein the compound is one of Formula IIIA or Formula IIB

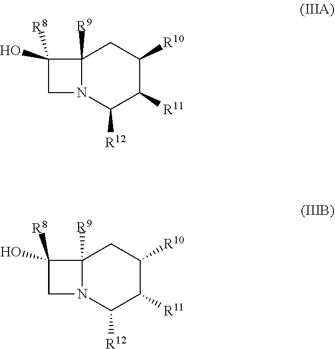

or a pharmaceutically acceptable salt and/or solvate thereof.
F. The compound of Paragraph A, wherein the compound is one of Formula VIA, Formula VIB, Formula VIC, or Formula VID

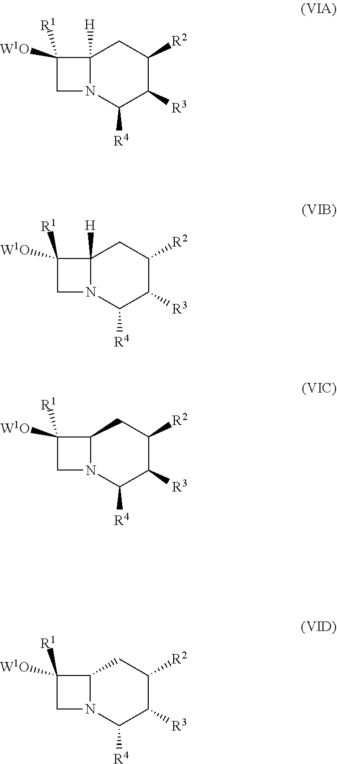

or a pharmaceutically acceptable salt and/or solvate thereof.
G. The compound of Paragraph A, wherein the compound is one of Formula VIIA, Formula VIIB, Formula VIIC, or Formula VIID

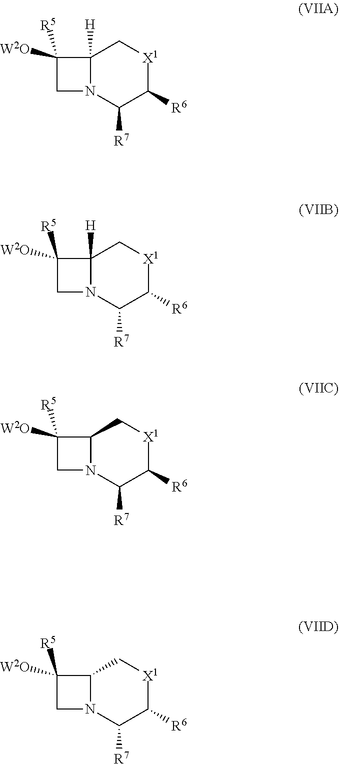

or a pharmaceutically acceptable salt and/or solvate thereof.
H. The compound of Paragraph A, wherein the compound is one of Formula VIIIA or Formula VIIB

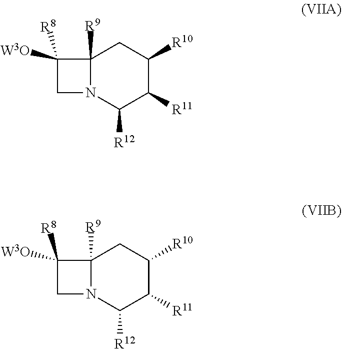

or a pharmaceutically acceptable salt and/or solvate thereof.
I. The compound of any one of Paragraphs A and F-H, wherein W¹, W², and W³ are independently at each occurrence

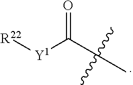

J. The compound of Paragraph M, wherein Y¹ is CH₂.
K. The compound of Paragraph M, wherein Y¹ is CN₂.
L. A compound of Formula IV

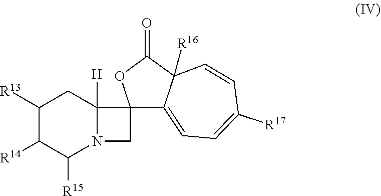

or a pharmaceutically acceptable salt and/or solvate thereof; wherein
   R¹³, R¹⁴, and R¹⁵ are each independently H, alkyl, or halo;
   R¹⁶ is aryl or heteroaryl; and
   R¹⁷ is H, alkyl, or halo.
M. The compound of Paragraph L, wherein the compound is one of Formula IVA or Formula IVB

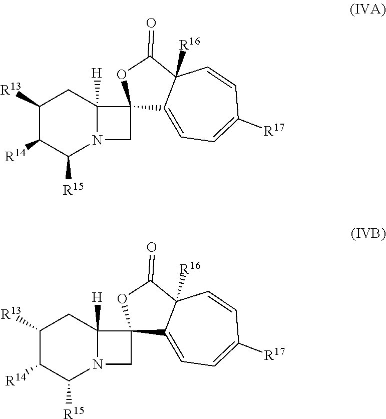

or a pharmaceutically acceptable salt and/or solvate thereof.
N. A compound of Formula V

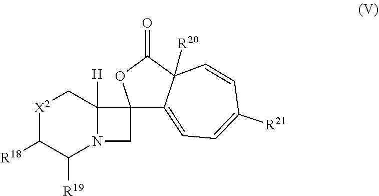

or a pharmaceutically acceptable salt and/or solvate thereof; wherein
   X² is O, NH, or S;
   R¹⁸ and R¹⁹ are each H, alkyl, or halo;
   R²⁰ is aryl or heteroaryl; and
   R²¹ is H, alkyl, or halo.
O. The compound of Paragraph N, wherein the compound is one of Formula VA or Formula VB

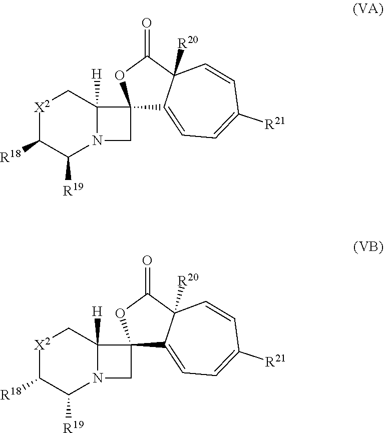

or a pharmaceutically acceptable salt and/or solvate thereof.
P. The compound of any one of Paragraphs A-O, wherein the compound is

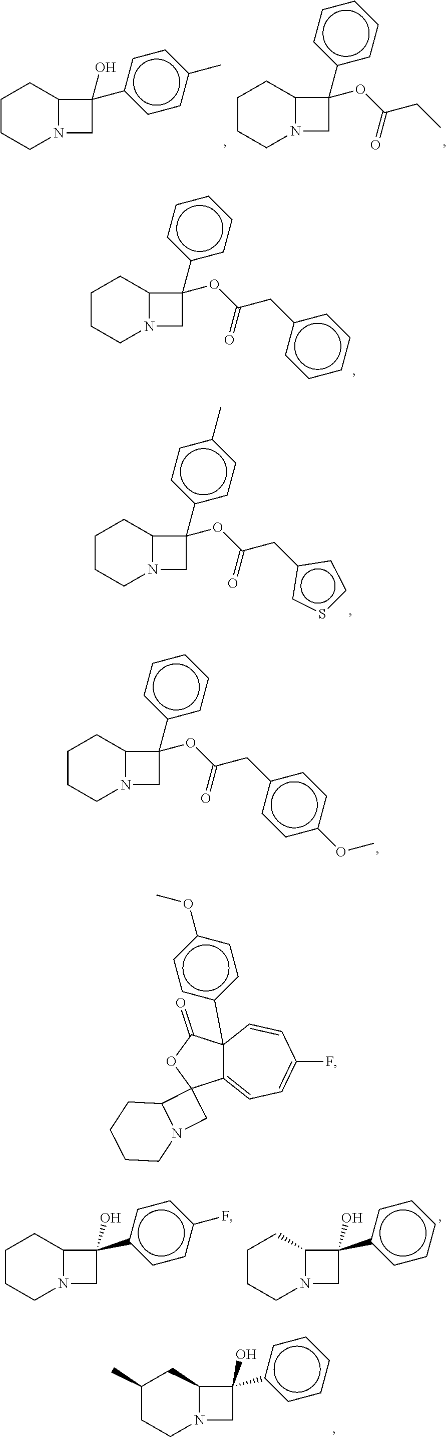

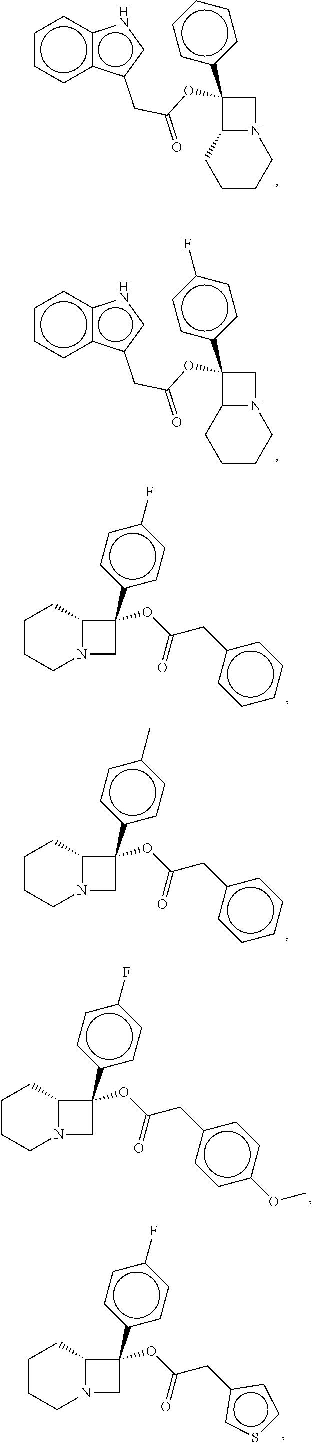

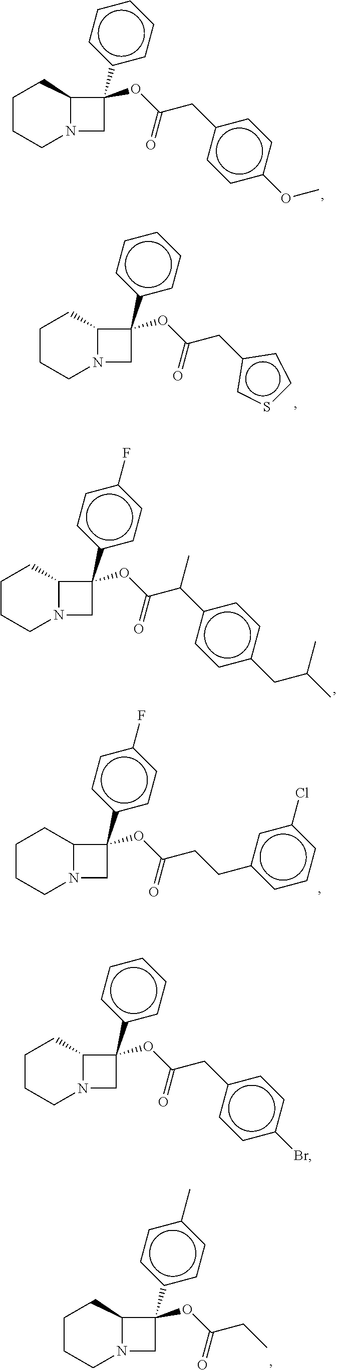

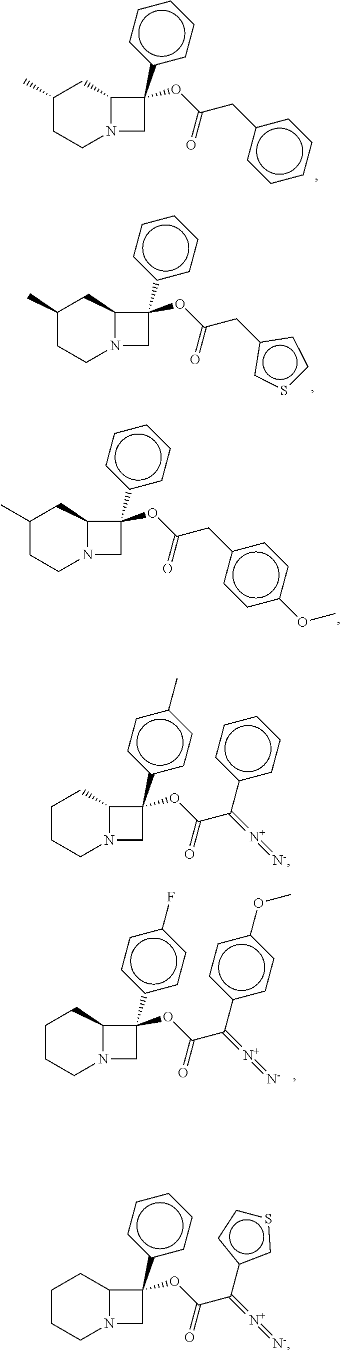

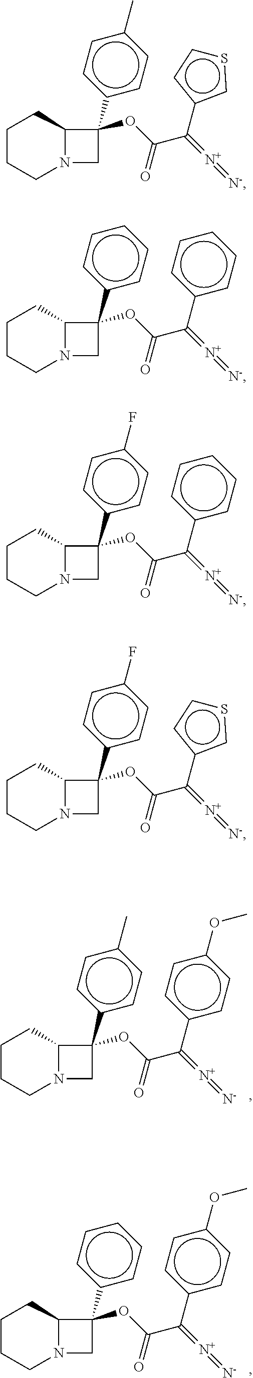

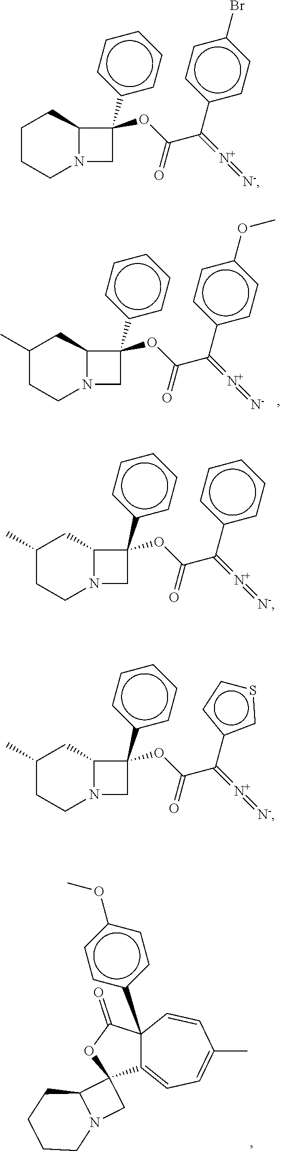

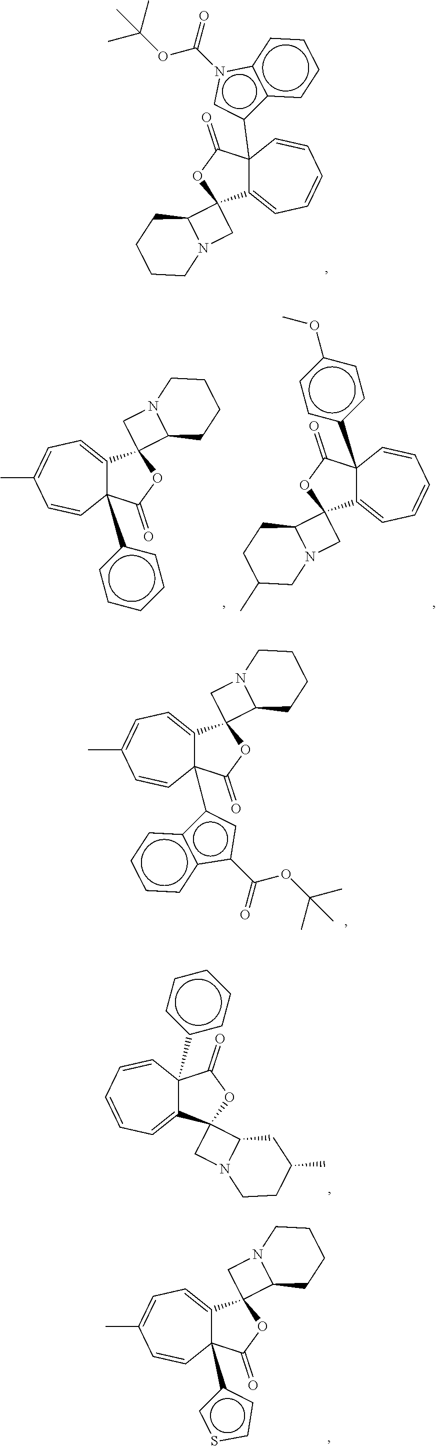

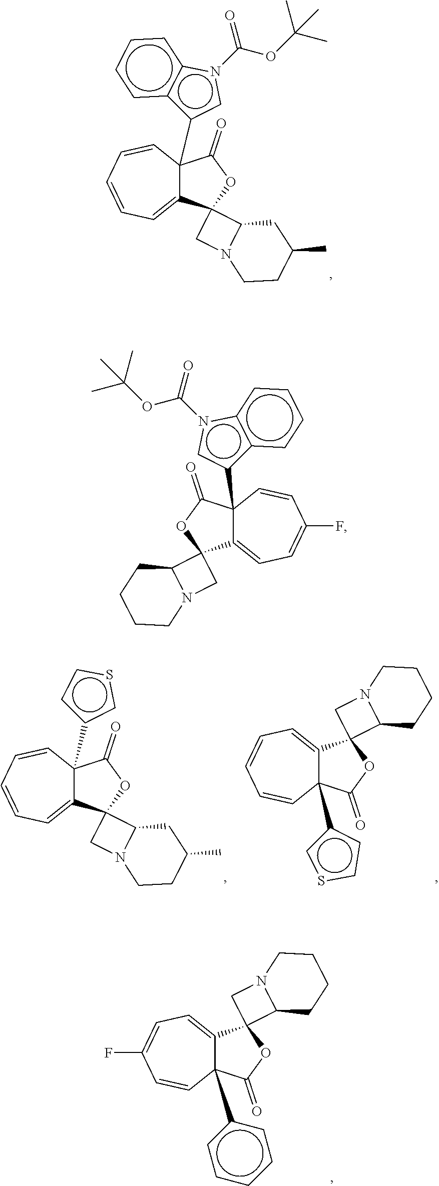

or pharmaceutically acceptable salt and/or solvate of any thereof.

### REFERENCES

[1] Nicolaou, K.; Vourloumis, D.; Winssinger, N.; Baran, P. S. The art and science of total synthesis at the dawn of the twenty-first century. Angewandte Chemie International Edition 2000, 39, 44-122.
[2] Burke, M. D.; Schreiber, S. L. A planning strategy for diversity-oriented synthesis. Angewandte Chemie International Edition 2004, 43, 46-58.
[3] Bertz, S. H. The first general index of molecular complexity. Journal of the American Chemical Society 1981, 103, 3599-3601.
[4] Böttcher, T. An Additive Definition of Molecular Complexity. J. Chem. Inf. Model. 2016, 56, 462-470.
[5] Demoret, R. M.; Baker, M. A.; Ohtawa, M.; Chen, S.; Lam, C. C.; Khom, S.; Roberto, M.; Forli, S.; Houk, K. N.; Shenvi, R. A. Synthetic, Mechanistic, and Biological Interrogation of Ginkgo biloba Chemical Space En Route to (−)-Bilobalide. Journal of the American Chemical Society 2020, 142, 18599-18618.
[6] Wagner, B. K.; Schreiber, S. L. The power of sophisticated phenotypic screening and modern mechanism-of-action methods. Cell chemical biology 2016, 23, 3-9.
[7] Breslow, R.; Winnik, M. A. Remote oxidation of unactivated methylene groups. Journal of the American Chemical Society 1969, 91, 3083-3084.
[8] Buchner, E.; Curtius, T. Ueber die Einwirkung von Diazoessigäther auf aromatische Kohlenwasserstoffe. Berichte der deutschen chemischen Gesellschaft 1885, 18, 2377-2379.
[9] Fleming, G. S.; Beeler, A. B. Regioselective and Enantioselective Intermolecular Buchner Ring Expansions in Flow. Organic letters 2017, 19, 5268-5271.
[10] Yang, N. C.; Yang, D.-D. H. Photochemical Reactions Of Ketones In Solution. Journal of the American Chemical Society 1958, 80, 2913-2914.
[11] Chen, C. The past, present, and future of the Yang reaction. Organic & biomolecular chemistry 2016, 14, 8641-8647.
[12] Baum, W. A.; Dunkelman, L. Ultraviolet Radiation of the High Pressure Xenon Arc. J. Opt. Soc. Am. 1950, 40, 782-786.
[13] Becker, M. R.; Wearing, E. R.; Schindler, C. S. Synthesis of azetidines via visible-light-mediated intermolecular [2+2] photocycloadditions. Nature Chemistry 2020, 12, 898-905.
[14] Becker, M. R.; Richardson, A. D.; Schindler, C. S. Functionalized azetidines via visible light-enabled aza Paternò-Büchi reactions. Nature communications 2019, 10, 1-8.
[15] Mitasev, B. et al. Crystallization-Based Synthetic Route to Antimalarial Agent BRD5018: Diazocene Ring Formation via a Staudinger-aza-Wittig Reaction on an Azetidine-Ribose Template. Organic Process Research & Development 2021,
[16] Ruggeri, M.; Dombrowski, A. W.; Djuric, S. W.; Baxendale, I. R. Photochemical Flow Synthesis of 3-Hydroxyazetidines. ChemPhotoChem 2019, 3, 1212-1218.
[17] Jurczyk, J.; Lux, M. C.; Adpressa, D.; Kim, S. F.; Lam, Y.-h.; Yeung, C. S.; Sarpong, R. Photomediated ring contraction of saturated heterocycles. Science 2021, 373, 1004-1012.
[18] Roberts, C. A.; Park, B.; Xu, L.-P.; Roque, J. B.; Yeung, C. S.; Musaev, D. G.; Sarpong, R.; LaLonde, R. L. Sequential Norrish-Yang Cyclization and C-C Cleavage/Cross-Coupling of a [4.1. 0] Fused Saturated Azacycle. The Journal of Organic Chemistry 2021, 86, 12436-12442.
[19] Eliel, E. L.; Wilen, S. H. Stereochemistry of organic compounds; John Wiley & Sons, 1994.
[20] Horspool, W. M.; Lenci, F. CRC Handbook of Organic Photochemistry and Photobiology, Volumes 1 & 2; CRC press, 2003; Chapter 52, pp 18,19.
[21] Small Jr, R.; Scaiano, J. Direct detection of the biradicals generated in the Norrish type II reaction. Chemical Physics Letters 1977, 50, 431-434.
[22] Calvert, J. G.; Pitts, J. N. Photochemistry. Wiley, New York 1966, 8, 99.
[23] Malinowski, E. R.; Howery, D. G. Factor analysis in chemistry; Wiley, 1980.
[24] Weismüller, J. A.; Chanady, A. Quantitative multicomponent analysis of complex mixtures by means of Full Spectrum quantitation and principal component analysis. TrAC Trends in Analytical Chemistry 1992, 11, 86-90.
[25] Hatchard, C.; Parker, C. A. A new sensitive chemical actinometer-II. Potassium ferrioxalate as a standard chemical actinometer. Proceedings of the Royal Society of London. Series A. Mathematical and Physical Sciences 1956, 235, 518-536.
[26] Kuhn, H.; Braslavsky, S.; Schmidt, R. Chemical actinometry (IUPAC technical report).
Pure and Applied Chemistry 2004, 76, 2105-2146.
[27] Johnson, R. C. Convenient procedure for the preparation of potassium trioxalatoferrate (III). Journal of Chemical Education 1970, 47, 702.
[28] Neises, B.; Steglich, W. Simple method for the esterification of carboxylic acids. Angewandte Chemie International Edition in English 1978, 17, 522-524.
[29] Baum, J. S.; Shook, D. A.; Davies, H. M.; Smith, H. D. Diazotransfer reactions with p-acetamidobenzenesulfonyl azide. Synthetic communications 1987, 17, 1709-1716.
[30] Lambert, J. B.; Durham, L. J.; Lepoutere, P.; Roberts, J. D. Nuclear Magnetic Resonance Spectroscopy. Analysis and Temperature Dependence of the Spectra of Cycloheptatriene and 7, 7-Bistrifluoromethylcycloheptatriene1. Journal of the American Chemical Society 1965, 87, 3896-3899.
[31] Karplus, M. Vicinal proton coupling in nuclear magnetic resonance. Journal of the American Chemical Society 1963, 85, 2870-2871.
[32] Haasnoot, C.; de Leeuw, F. A.; Altona, C. The relationship between proton-proton NMR coupling constants and substituent electronegativities—I: An empirical generalization of the karplus equation. Tetrahedron 1980, 36, 2783-2792.
[333] Jurberg, I. D.; Davies, H. M. Blue light-promoted photolysis of aryldiazoacetates. Chemical science 2018, 9, 5112-5118.

What is claimed is:

1. A compound of Formula IV 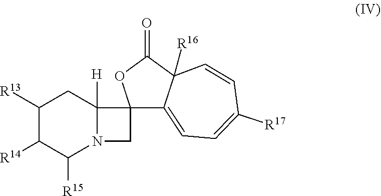
or a pharmaceutically acceptable salt and/or solvate thereof; wherein
R¹³, R¹⁴, and R¹⁵ are each independently H, alkyl, or halo;
R¹⁶ is aryl or heteroaryl; and
R¹⁷ is H, alkyl, or halo.

2. The compound of claim 1, wherein the compound is one of Formula IVA or Formula IVB 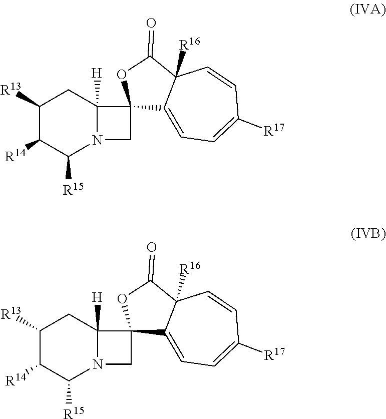
or a pharmaceutically acceptable salt and/or solvate thereof.

3. A compound of Formula V 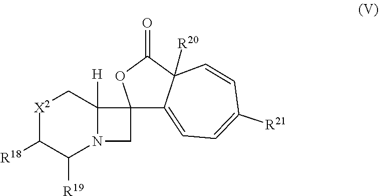
or a pharmaceutically acceptable salt and/or solvate thereof; wherein
X² is O, NH, or S;
R¹⁸ and R¹⁹ are each H, alkyl, or halo;
R³⁰ is aryl or heteroaryl; and
R²¹ is H, alkyl, or halo.

4. The compound of claim 3, wherein the compound is one of Formula VA or Formula VB 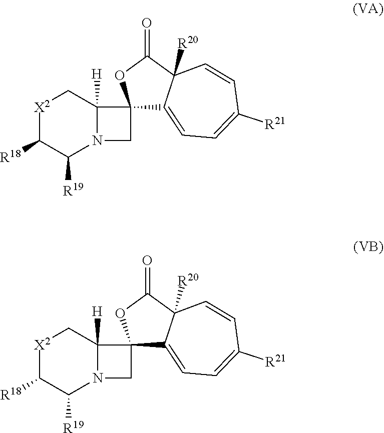
or a pharmaceutically acceptable salt and/or solvate thereof.

5. A compound of Formula I, Formula II, or Formula III 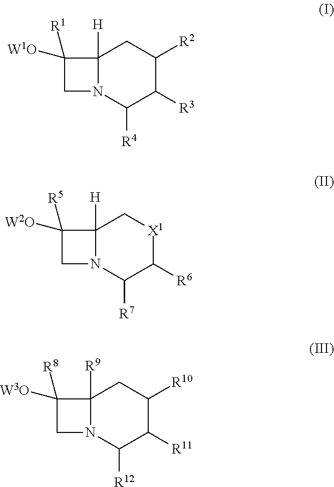
or a pharmaceutically acceptable salt and/or solvate thereof; wherein
R¹ is aryl or heteroaryl;
R², R³, and R⁴ are each independently H, alkyl, or halo;
R⁵ is aryl or heteroaryl;
X¹ is O, NH, or S;
R⁶ and R⁷ are each independently H, alkyl, or halo;
R⁸ is aryl or heteroaryl;
R⁹ is alkyl;
R¹⁰, R¹¹, and R¹² are each independently H, alkyl, or halo; and
W¹, W², and W³ are independently at each occurrence H or 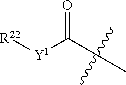
wherein Y¹ is CH₂ or CN₂, and R²² is alkyl, aryl, or heteroaryl.

6. The compound of claim 5, wherein W¹, W², and W³ are independently at each occurrence H.

7. The compound of claim 5, wherein the compound is one of Formula IA, Formula IB, Formula IC, or Formula ID 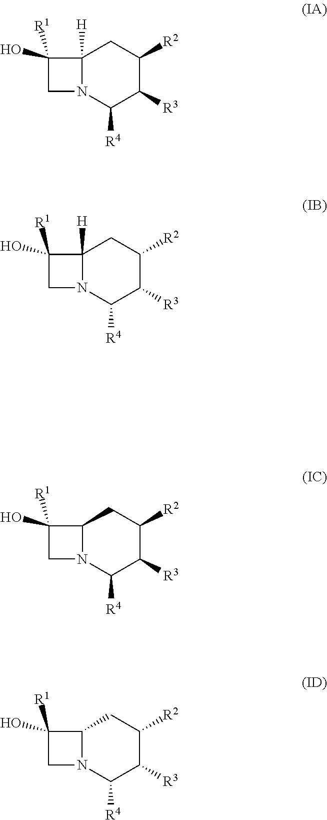
or a pharmaceutically acceptable salt and/or solvate thereof.

8. The compound of claim 5, wherein the compound is one of Formula IIA, Formula IIB, Formula IIC, or Formula IID 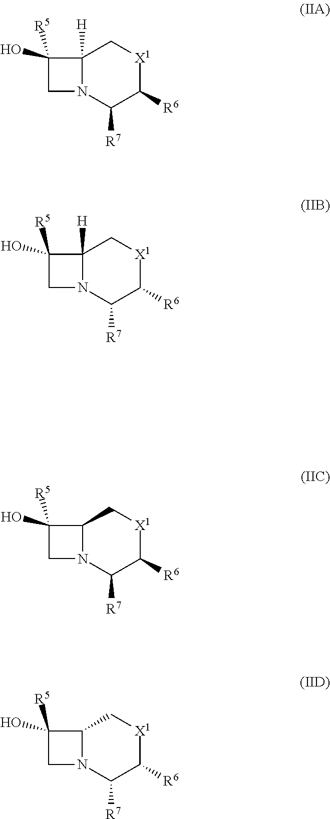
or a pharmaceutically acceptable salt and/or solvate thereof.

9. The compound of claim 5, wherein the compound is one of Formula IIIA or Formula IIB 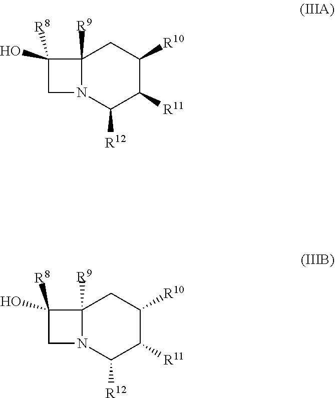
or a pharmaceutically acceptable salt and/or solvate thereof.

10. The compound of claim 5, wherein the compound is one of Formula VIA, Formula VIB, Formula VIC, or Formula VID 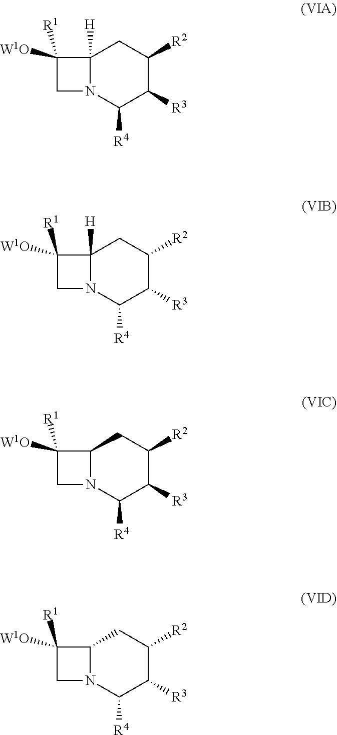
or a pharmaceutically acceptable salt and/or solvate thereof.

11. The compound of claim 5, wherein the compound is one of Formula VIIA, Formula VIB, Formula VIIC, or Formula VIID 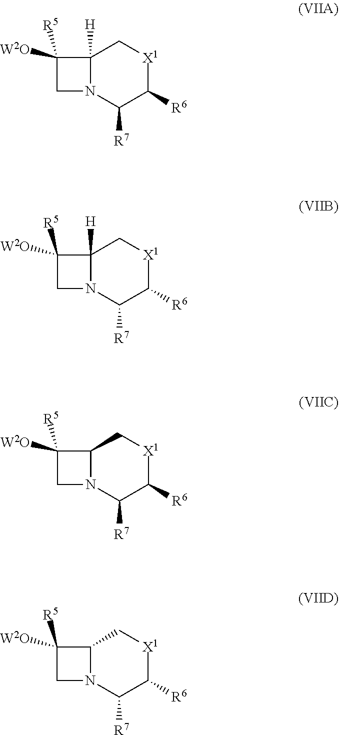
or a pharmaceutically acceptable salt and/or solvate thereof.

12. The compound of claim 5, wherein the compound is one of Formula VIIIA or Formula VIIIB 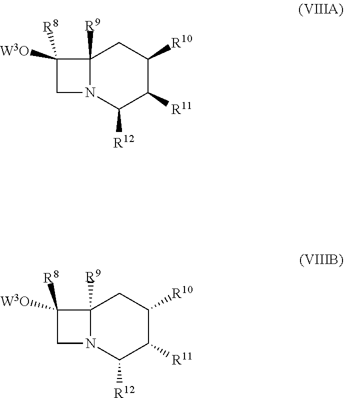
or a pharmaceutically acceptable salt and/or solvate thereof.

13. The compound of claim 10, wherein W¹, W², and W³ are independently at each occurrence

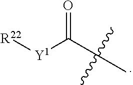

14. The compound of claim 13, wherein Y¹ is CH₂.

15. The compound of claim 13, wherein Y¹ is CN₂.

16. The compound of claim 11, wherein W¹, W², and W³ are independently at each occurrence

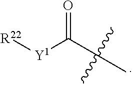

17. The compound of claim 16, wherein Y¹ is CH₂.

18. The compound of claim 16, wherein Y¹ is CN₂.

19. The compound of claim 12, wherein W¹, W², and W³ are independently at each occurrence

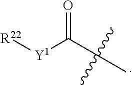

20. The compound of claim 16, wherein Y¹ is CH₂.
